(19) United States
(12) Patent Application Publication
(10) Pub. No.: US 2015/0336948 A1
(43) Pub. Date: Nov. 26, 2015

(54) Isotopically Labeled Biaryl Urea Compounds

(71) Applicant: Merck Sharp & Dohme Corp., Rahway, NJ (US)

(72) Inventors: Michael D. Altman, Needham, MA (US); Christian Fischer, Needham, MA (US); Jason D. Katz, Newton, MA (US); Theresa M. Williams, Harleysville, PA (US); Xu-Fang Zhang, Dresher, PA (US); Hua Zhou, Walgham, MA (US)

(21) Appl. No.: 14/404,208

(22) PCT Filed: May 24, 2013
(86) PCT No.: PCT/US13/42554

§ 371 Date: Nov. 26, 2014

Related U.S. Application Data
(60) Provisional application No. 61/652,554, filed on May 29, 2012.

(51) Int. Cl. C07D 471/04; C07D 231/12

(52) U.S. Cl. CPC C07D 471/04; C07D 231/12

(57) ABSTRACT

The present invention is directed to isotopically labeled biaryl urea compounds which possess high affinity to neurofibrillary tangles (NFTs), and thus are useful to determine the amount and distribution of NFTs in brain. The isotopically labeled biaryl urea compounds may also be useful as PET tracers and in competition assays to identify other compounds that may serve as PET tracers.

### BACKGROUND OF THE INVENTION

It is well established that Alzheimer's disease and a number of related tauopathies including Pick's disease, Corticobasal Degeneration and Progressive Supranuclear Palsy are characterized, in part, by the development of neurofibrillary tangles (NFTs). These NFTs are aggregated filaments, of either paired helical filaments (PHFs) (as in Alzheimer's Disease) or straight filaments (as in Progressive Supranuclear Palsy) composed of the microtubule associated protein tau (tau). Normally tau stabilizes a key cellular network of microtubules that is essential for distributing proteins and nutrients within neurons. In Alzheimer's disease patients, however, tau becomes hyperphosphorylated, disrupting its normal functions, increasing its likelihood to aggregate and ultimately forming neurofibrillary lesions, such as NFTs. Six isoforms of tau are found in the human brain. In Alzheimer's disease patients, all six isoforms of tau are found in NFTs, and all are markedly hyperphosphorylated (Goedert et al., Neuron 1992, 8, 159; and Goedert et al., Neuron 1989, 3, 519).

Tau in healthy brain tissue bears only 2 or 3 phosphate groups, whereas those found in the brains of Alzheimer's disease subjects bear, on average, 8 phosphate groups (Kopke et al., J Biol Chem 1993, 268, 24374; and Ksiezak-Reding et al., Brain Res 1992, 597, 209). A clear parallel between NFT levels in the brains of Alzheimer's disease patients, location of neurodegeneration and the severity of dementia strongly supports a key role for tau dysfunction in Alzheimer's disease (Henrissat et al., Biochem J 1996, 316 (Pt 2), 695; Henrissat et al., Biochem J 1993, 293 (Pt 3), 781; Gomez-Isla et al., J. Neuroscience, 1996, 16(14), 4491-4500)); and Arriagada, et al, Neurology 1992, 42, 631-639).

Noninvasive nuclear imaging techniques can be used to obtain basic and diagnostic information about the physiology and biochemistry of a variety of living subjects including experimental animals, normal humans and patients. These techniques rely on the use of sophisticated imaging instrumentation that is capable of detecting radiation emitted from radiotracers administered to such living subjects. The information obtained can be reconstructed to provide planar and tomographic images that reveal distribution of the radiotracer as a function of time. Use of appropriately designed radiotracers can result in images which contain information on the structure, function and most importantly, the physiology and biochemistry of the subject. Much of this information cannot be obtained by other means. The radiotracers used in these studies are designed to have defined behaviors in vivo which permit the determination of specific information concerning the physiology or biochemistry of the subject or the effects that various diseases or drugs have on the physiology or biochemistry of the subject. Currently, radiotracers are available for obtaining useful information concerning such things as cardiac function, myocardial blood flow, lung perfusion, liver function, brain blood flow, brain regional distribution and function.

For noninvasive in vivo imaging, compounds can be labeled with either positron- or gamma-emitting isotopes. The most commonly used positron emitting (PET) isotopes are ¹¹C, ¹⁸F, ¹⁵O and ¹³N, all of which are accelerator produced, and have half-lives of 20, 110, 2 and 10 minutes, respectively. These short half-lives endow a number of advantages to their use as tracers to probe biological processes in vivo using PET. Since the half-lives of these isotopes are so short, it is only feasible to use them at institutions that have an accelerator on site or very close by for their production, thus limiting their use.

In a typical PET study, a small amount of radiotracer is administered to the experimental animal, normal human or patient being tested. The radiotracer then circulates in the blood of the subject and may be absorbed in certain tissues. The radiotracer may be preferentially retained in some of these tissues because of specific enzymatic conversion or by specific binding to macromolecular structures such as proteins. Using sophisticated imaging instrumentation to detect photons resulting from positron emission, the amount of radiotracer is then non-invasively assessed in the various tissues in the body. The resulting data are analyzed to provide quantitative spatial information of the in vivo biological process for which the tracer was designed. PET gives pharmaceutical research investigators the capability to assess biochemical changes or metabolic effects of a drug candidate in vivo for extended periods of time, and PET can be used to measure drug distribution, thus allowing the evaluation of the pharmacokinetics and pharmacodynamics of a particular drug candidate under study. Importantly, PET tracers can be designed and used to quantitate the presence of binding sites in tissues. Consequently, interest in PET tracers for drug development has been expanding based on the development of isotopically labeled biochemicals and appropriate detection devices to detect the radioactivity by external imaging.

Noninvasive nuclear imaging techniques such as PET have been particularly important in providing the ability to study neurological diseases and disorders, including stroke, Parkinson's disease, epilepsy, cerebral tumors and Alzheimer's disease. A hallmark of Alzheimer's disease pathology is the presence of NFTs (Alzheimer, A., J. Gen. Psychiatr. (German, 1907, 64, 146-148; and Iqbal et al, J. Biol Chem, 1986, 261, 6084-9) and a PET radiotracer specific for NFTs would provide a powerful tool in demonstrating pharmacodynamic activity of therapies targeting reduction in NFTs by measuring changes in NFT levels and determining optimal doses in preclinical evaluation and clinical trials.

Disclosed herein are compounds possessing high affinity for NFTs and which when isotopically labeled are useful to study the distribution and abundance of NFT deposits in brain tissue samples. Such isotopically labeled compounds may also be useful in diagnostic imaging applications, e.g., non invasive in vivo imaging such as PET. Such isotopically labeled compounds would also be useful in competition assays to identify other compounds possessing high affinity for NFTs that may also be useful as PET tracers.

### SUMMARY OF THE INVENTION

The invention is directed to isotopically labeled biaryl urea compounds which bind with high affinity to NFTs in brain. The invention is also concerned with methods for the use of the isotopically labeled compounds for non invasive in vivo imaging such as PET. The invention is also concerned with the use of the isotopically labeled compounds to identify other compounds that possess high affinity for NFTs and thus, identify additional compounds that have potential as PET tracers for imaging NFTs in brain.

### BRIEF DESCRIPTION OF THE DRAWINGS

FIGS. 1A and 1B. Determination of binding site densities and binding affinity of [³H] 1-(2-methoxyphenyl)-3-(6-(1-methyl-1H-pyrrolo[2,3-c]pyridin-3-yl)pyridin-3-yl)urea (Example 7 compound) (FIG. 1A) and [³H] 1-(2-methoxyphenyl)-3-(4-(1-methyl-1H-pyrrolo[2,3-c]pyridin-3-yl)phenyl)urea (Example 6 compound) (FIG. 1B) to in vitro assembled tau filaments by hot saturation binding assay. Bmax and Kd values, calculated by non-linear regression, are expressed in nM.

FIG. 2. Displacement Binding Assay with In Vitro Assembled Tau Filaments. Determination of potency of 1-(2-methoxyphenyl)-3-(4-(1-methyl-1H-pyrrolo[2,3-c]pyridin-3-yl)phenyl)urea (Example 1 compound) to inhibit [³H] 1-(2-methoxyphenyl)-3-(4-(1-methyl-1H-pyrrolo[2,3-c]pyridin-3-yl)phenyl)urea (Example 6 compound) binding in vitro assembled tau filaments by the in vitro competition binding assay.

FIGS. 3A-3D. FIG. 3A is an autoradiograph from 5 nM [³H] 1-(2-methoxyphenyl)-3-(4-(1-methyl-1H-pyrrolo[2,3-c]pyridin-3-yl)phenyl)urea (Example 6 compound) binding; FIGURES B, C and D are immunohistochemical figures from PHF6 stain NFTs are shown in the hippocampus region by PHF6 stain, consistent with the autoradiographic binding pattern of [³H] 1-(2-methoxyphenyl)-3-(4-(1-methyl-1H-pyrrolo[2,3-c]pyridin-3-yl)phenyl)urea (Example 6 compound) in the adjacent slice. The bar to the right of the figures shows the relative optic density scale from low to high, corresponding to visual observation of tracer binding densities of the image.

FIG. 4 shows autoradiograph (ARG) and immunohistochemistry (IHC) images of human Alzheimer's disease brain cortex. FIG. 4A, lack of [³H] 1-(2-methoxyphenyl)-3-(4-(1-methyl-1H-pyrrolo[2,3-c]pyridin-3-yl)phenyl)urea (Example 6 compound) binding to amyloid plaques in cortex region. FIG. 4B, the adjacent human Alzheimer's disease brain cortex slice shows positive stain of dense amyloid plaques by IHC using 6E10 antibody.

FIGS. 5A and 5B. Determination of binding site densities and binding affinity of [³H] 1-(2-methoxyphenyl)-3-(6-(1-methyl-1H-pyrrolo[2,3-c]pyridin-3-yl)pyridin-3-yl)urea (Example 7 compound) and [³H] 1-(2-methoxyphenyl)-3-(4-(1-methyl-1H-pyrrolo[2,3-c]pyridin-3-yl)phenyl)urea (Example 6 compound) in human brain homogenates by in vitro hot saturation binding assay.

FIG. 6. Determination of potency of 1-(2-methoxyphenyl)-3-(6-(1-methyl-1H-pyrrolo[2,3-c]pyridin-3-yl)pyridin-3-yl)urea (Example 4 compound) to inhibit [³H] 1-(2-methoxyphenyl)-3-(6-(1-methyl-1H-pyrrolo[2,3-c]pyridin-3-yl)pyridin-3-yl)urea (Example 7 compound) binding in human Alzheimer's disease brain homogenates by in vitro competition binding assay.

### DETAILED DESCRIPTION OF THE INVENTION

As used above, and throughout this disclosure, the following terms, unless otherwise indicated, shall be understood to have the following meanings:

“One or more” means at least one.

“Subject” means an animal, such as a mammal, e.g., a rodent, non-human primate or a human.

“Isotopically labeled”, “tracer”, or “labeled tracer” compound, refers to a compound where one or more atoms are replaced or substituted by an atom having an atomic mass or mass number different from the atomic mass or mass number typically found in nature (i.e., naturally occurring). Suitable isotopes that may be incorporated in compounds of the present invention include, e.g., ²H, ³H, ¹¹C, ¹³, ¹⁴C, and ¹⁸F.

“Radioligand” or “radiotracer” refers to an isotopically labeled compound that is labeled with a radioactive isotope, e.g., ³H, ¹¹C, ¹⁴C and ¹⁸F.

“Effective amount” includes amounts that enable measuring/imaging of NFTs in vivo (i.e., diagnostically effective amount) that yield acceptable toxicity and bioavailability levels for pharmaceutical use, and also includes amounts that enable detection of NFTs in vitro, e.g., in brain tissue samples and in tau filaments.

This invention provides compounds having the Formula (I):

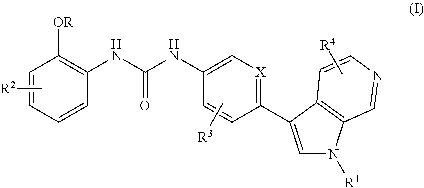

or a pharmaceutically acceptable salt thereof,
wherein

### X is N or C;

R is H or C1-6alkyl optionally substituted with one fluoro;
R¹ is H or C1-6alkyl optionally substituted with one fluoro; and
R², R³ and R⁴ are each independently H, fluoro or C1-6alkyl optionally substituted with one fluoro.

The compounds of the invention possess high affinity and selectivity for NFTs and thus are useful to study the regional distribution and concentration of NFTs in vitro in brain tissue samples. The compounds of the invention may also be utilized as PET tracers for imaging NFTs in the brain of living humans and experimental animals, i.e., determining the abundance and distribution of NFTs. Imaging of NFTs, in turn can aid in the diagnosis of a neurodegenerative disease associated with development of NFTs, e.g., Alzheimer's disease, as well as assess the progression and regression of such a neurodegenerative disease. Imaging of NFTs can also aid in assessing the effectiveness of various tau-directed therapies on the abundance and distribution of NFTs in brain. The compounds of the invention are also useful in competition assays, to identify other compounds that may be used as PET tracers for imaging NFTs in the brain of living humans and experimental animals.

In an embodiment of the compounds or a pharmaceutically acceptable salt thereof, R is methyl.

In another embodiment of the compounds or a pharmaceutically acceptable salt thereof, R is H.

In another embodiment of the compounds or pharmaceutically acceptable salt thereof, R¹ is methyl and R² is hydrogen.

In another embodiment of the compounds or pharmaceutically acceptable salt thereof, R², R³ and R⁴ are each H.

In another embodiment, the compounds are selected from the group consisting of

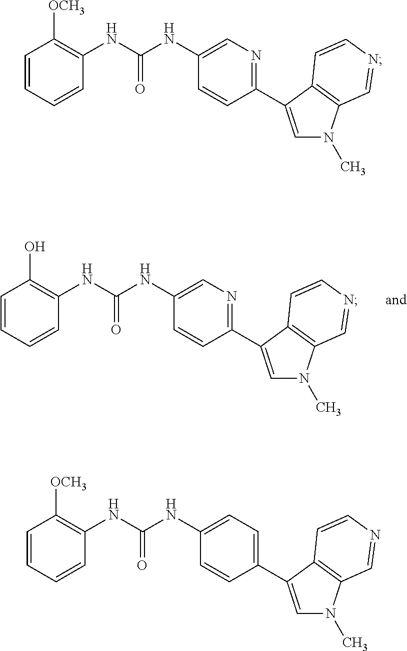 [SMILES: COc1ccccc1NC(=O)Nc1ccc(-c2cn(C)c3cnccc23)nc1 ; Cn1cc(-c2ccc(NC(=O)Nc3ccccc3O)cn2)c2ccncc21 ; "and" ; COc1ccccc1NC(=O)Nc1ccc(-c2cn(C)c3cnccc23)cc1]

or a pharmaceutically acceptable salt thereof.

In another embodiment, the invention includes isotopically labeled compounds and pharmaceutically acceptable salts thereof. Suitable isotopes that may be incorporated in compounds of the invention include but are not limited to ²H, ₃H, ¹¹C, ¹³C, ¹⁴C, and ¹⁸F and preferably ³H. The isotopically labeled compounds of the invention need only to be enriched with an isotope to, or above, the degree which allows detection with a technique suitable for the particular application. The isotope that is incorporated in the instant isotopically labeled compounds will depend on the specific application of that isotopically labeled compound.

In another embodiment, the isotopically labeled compound of Formula (I) is:

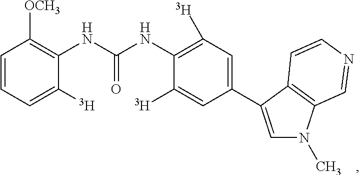 [SMILES: [3H]c1cc(-c2cn(C)c3cnccc23)cc([3H])c1NC(=O)Nc1c([3H])cccc1OC]

or a pharmaceutically acceptable salt thereof.

In another embodiment, the isotopically labeled compound of Formula (I) is:

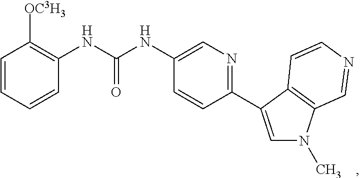

or a pharmaceutically acceptable salt thereof.

The invention also provides for compounds having the Formula (II)

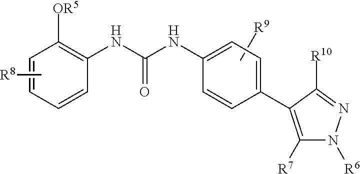

or a pharmaceutically acceptable salt thereof, wherein
R⁵ is H or C1-6alkyl optionally substituted with one fluoro;
R⁶ is H or C1-6alkyl optionally substituted with one fluoro;
R⁷ is H, fluoro or C1-6alkyl optionally substituted with one fluoro; or
R⁶ and R⁷ together complete a 5-6-membered saturated heterocyclic ring containing 4-5 carbon atoms; and
R⁸, R⁹ and R¹⁰ are each independently H, fluoro or C1-6alkyl optionally substituted with one fluoro.

In an embodiment of the compounds, R⁵ is methyl.

In another embodiment of the compounds, R⁶ is hydrogen and R⁷ is methyl.

In another embodiment of the compounds, R⁶ and R⁷ together complete a 5-6 membered saturated heterocyclic ring, e.g., pyrrolidinyl or piperidinyl.

In another embodiment of the compounds, the saturated heterocyclic ring formed from R⁶ and R⁷ is pyrrolidinyl.

In another embodiment of the compounds of Formula (II) or a pharmaceutically acceptable salt thereof, the compounds can be isotopically labeled with an isotope selected from the group consisting of ²H, ³H, ¹¹C, ¹³C, ¹⁴C, and ¹⁸F, and preferably ³H.

In another embodiment, the compounds are selected from the group consisting of

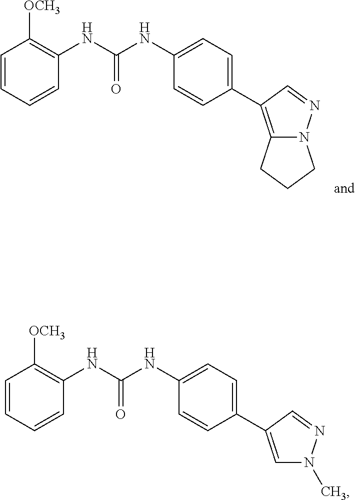 [SMILES: COc1ccccc1NC(=O)Nc1ccc(-c2cnn3c2CCC3)cc1 ; COc1ccccc1NC(=O)Nc1ccc(-c2cnn(C)c2)cc1]

or a pharmaceutically acceptable salt thereof.

The compounds of Formulas (I) and (II) may have asymmetric centers, chiral axes and chiral planes, and occur as racemates, racemic mixtures, and as individual diastereomers, with all possible isomers, including optical isomers, being included in the present invention. (See E. L. Eliel and S. H. Wilen Stereochemistry of Carbon Compounds (John Wiley and Sons, New York 1994), in particular pages 1119-1190).

Salts of the compounds of Formulas (I) and (II) will be pharmaceutically acceptable salts. Other salts may, however, be useful in the preparation of the compounds according to the invention or of their pharmaceutically acceptable salts. When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

Salts of the compounds which are in basic form may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The preparation of the pharmaceutically acceptable salts described above and other typical pharmaceutically acceptable salts is more fully described by Berg et al., “Pharmaceutical Salts,” J. Pharm. Sci., 1977:66:1-19.

The invention also provides a method for the detection or quantification of NFTs in mammalian brain tissue, the method comprising contacting the mammalian tissue in which such detection is desired with an effective amount of the isotopically labeled compound selected from the group consisting of the compounds of Examples 6 and 7 or a pharmaceutically acceptable salt thereof.

In an embodiment of the aforementioned methods of the invention, the mammal is e.g., a rodent, a non-human primate or a human.

In another embodiment of the aforementioned methods of the invention, detection or quantification of NFTs in brain is carried out by performing PET imaging, magnetic resonance imaging, or autoradiography.

Isotopically labeled compounds of the invention are potentially useful for diagnostic imaging or basic research applications. Specific examples of possible diagnostic imaging and basic research applications, include determining the location or abundance of NFTs in brain, and autoradiography to determine the distribution of NFTs in the brain of a mammal.

In particular, these isotopically labeled compounds, when labeled with the positron emitting radionuclide, ¹⁸F, may be useful for PET imaging of NFTs in the brain of living humans and experimental animals. The isotopically labeled compounds may be used as research tools to determine the location and level of NFTs in the brain and to determine changes in NFTs levels as a result of treatment with compounds that effect the levels of NFTs in the brain working through a variety of molecular targets. In animal experiments, these isotopically labeled compounds can be used to provide information that is useful for choosing between potential drug candidates for selection for clinical development by differentiating compounds based on their ability to lower NFT levels in the brain. The isotopically labeled compounds of the invention may also be used to study the regional distribution and concentration of NFTs in the living human brain, as well as the brain of living experimental animals and in tissue samples. The isotopically labeled compounds may also be used to study disease or pharmacologically related changes in NFT concentrations. For example, PET tracers such as the present isotopically labeled compounds may be used with currently available PET technology as a tool to diagnose Alzheimer's disease in subjects as well as assess the progression or regression of Alzheimer's disease in subjects. The present isotopically labeled compounds may also have use in assessing the efficacy of various tau-targeted therapies, e.g., tau aggregation inhibitors, tau phosphorylation inhibitors, and microtubule stabilizers on in vivo density and distribution of NFTs during the treatment of Alzheimer's disease with such tau-targeted therapies.

It is well established that Alzheimer's disease and a number of related tauopathies as described below are characterized, in part, by the development of NFTs and thus, the isotopically labeled compounds of the invention may also have utility in diagnostic imaging with respect to a variety of neurological and psychiatric disorders associated with NFT formation including Alzheimer's disease as discussed above and related tauopathies. Related tauopathies include but are not limited to, Corticobasal Degeneration, Progressive Supranuclear Palsy, Argyrophilic grain dementia, Dementia pugilistica, Diffuse neurofibrillary tangles with calcification, Familial British dementia, Familial Danish dementia, Frontotemporal dementia with parkinsonism linked to chromosome 17 (FTDP-17), Gerstmann-Straussler-Scheinker disease, Guadeloupean parkinsonism, Hallevorden-Spatz disease (neurodegeneration with brain iron accumulation type 1), Inclusion body Myositis, Multiple system atrophy, Myotonic dystrophy, Niemann-Pick disease (type C), Pallido-ponto-nigral degeneration, Amyotrophic lateral sclerosis/parkinsonism-dementia complex of Guam, Pick's disease, Post-encephalitic parkinsonism, Prion diseases (including Creutzfeldt-Jakob Disease, Variant Creutzfeldt-Jakob Disease, Fatal Familial Insomnia, and Kuru), Progressive supercortical gliosis, Richardson's syndrome, Subacute sclerosing panencephalitis, and Tangle-only dementia.

For the use of the instant compounds as exploratory or diagnostic imaging agents the isotopically labeled compounds may be administered to mammals, preferably humans, in a pharmaceutical composition, either alone or, preferably, in combination with one or more pharmaceutically acceptable carriers or diluents, optionally with known adjuvants, such as alum, in a pharmaceutical composition, according to standard pharmaceutical practice. Such compositions can be administered orally or parenterally, including the intravenous, intramuscular, intraperitoneal, subcutaneous, rectal and topical routes of administration. Preferably, administration is intravenous. Radiotracers labeled with short-lived, positron emitting radionuclides are generally administered via intravenous injection within less than one hour of their synthesis. This is necessary because of the short half-life of the isotopes involved (20 and 110 minutes for ¹¹C and ¹⁸F, respectively).

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and one or more pharmaceutically acceptable carriers. By “pharmaceutically acceptable” it is meant the carrier, diluent or excipient must be compatible with the other ingredients of the formulation and not deleterious to the recipient thereof. The terms “administration of” and or “administering a” compound should be understood to mean providing a compound of the invention, or pharmaceutically acceptable salt or in vivo hydrolysable ester thereof to the subject. The pharmaceutical compositions of this invention may be used in the form of a pharmaceutical preparation, for example, in solid, semisolid or liquid form, which contains one or more of the compounds of the present invention, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used are water, glucose, lactose, gum acacia, gelatin, mannitol, starch paste, magnesium trisilicate, talc, corn starch, keratin, colloidal silica, potato starch, urea and other carriers suitable for use in manufacturing preparations, in solid, semisolid, or liquid form, and in addition auxiliary, stabilizing, thickening and coloring agents and perfumes may be used. The active object compound is included in the pharmaceutical composition in an amount sufficient to produce the desired effect upon the process or condition of the disease.

The liquid forms in which the novel compositions of the present invention may be incorporated for administration orally or by injection include aqueous solution, suitably flavoured syrups, aqueous or oil suspensions, and emulsions with acceptable oils such as cottonseed oil, sesame oil, coconut oil or peanut oil, or with a solubilizing or emulsifying agent suitable for intravenous use, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

When an isotopically labeled compound according to this invention is administered into a human subject, the amount required for diagnostic imaging will normally be determined by the prescribing physician with the dosage generally varying according to the age, weight, and response of the individual subject, as well as the quantity of emission from the radionuclide. However, in most instances, an effective amount will be the amount of compound sufficient to produce emissions in the range of from about 1-10 mCi.

In one exemplary application, administration occurs in an amount of isotopically labeled compound of between about 0.005 μg/kg of body weight to about 50 μg/kg of body weight per day, preferably of between 0.02 μg/kg of body weight to about 7 μg/kg of body weight. A particular analytical dosage that comprises the instant composition includes from about 0.5 μg to about 100 μg of the isotopically labeled compound. Preferably, the dosage comprises from about 1 μg to about 50 μg of the isotopically labeled compound.

The following illustrative procedure may be utilized when performing PET imaging studies on subjects in the clinic. The subject undergoes a baseline scan as described below, after which the subject is premedicated with unlabeled compound of the present invention for the desired time prior to the day of the experiment and is fasted for at least 12 hours allowing water intake ad libitum. A 20 G two inch venous catheter is inserted into the contralateral ulnar vein for radiotracer administration.

The subject is positioned in a supine position in the PET camera and a sufficient amount (about 1-10 mCi) of an isotopically labelled tracer is administered to the subject. An emission scan of the cerebral region is performed. The technique for performing an emission scan of the head is well known to those skilled in the art. PET techniques are described in Freeman et al., Freeman and Johnson's Clinical Radionuclide Imaging. 3^{rd}. Ed. Vol. 1 (1984); Grune & Stratton, New York; Ennis et al., Vascular Radionuclide Imaging: A Clinical Atlas, John Wiley & Sons, New York (1983). For determining the distribution of radiotracer, regions of interest are drawn on the reconstructed image including, e.g. the brain and the central nervous system. These regions are used to generate time activity curves obtained under baseline conditions and after treatment with a tau-directed therapy. Kinetic modeling as applied by those skilled in the art, is then used to determine changes in cerebral NFT levels.

The invention is also, in part, directed to a method for identifying compounds that can be used as PET tracers, e.g., in displacement binding assays. In one embodiment, the method comprises contacting tau filaments with an isotopically labeled compound as described herein and then determining the amount of displaced binding of the isotopically labeled compound in the presence and absence of the compound of interest.

In accordance with another embodiment of the present invention, there are provided methods for the preparation of compounds of invention as described below. For example, the compounds can be prepared using synthetic chemistry techniques well known in the art (see Comprehensive Heterocyclic Chemistry, Katritzky, A. R. and Rees, C. W. eds., Pergamon Press, Oxford, 1984) from a precursor of the compounds as outlined below. The isotopically labeled compounds of this invention are prepared by incorporating the aforementioned isotopes, e.g., into the substrate molecule. This is accomplished by utilizing reagents that have had one or more of the atoms contained therein made radioactive by placing them in a source of radioactivity such as a nuclear reactor, a cyclotron and the like. Additionally many isotopically labeled reagents, such as ²H₂O, ³H₃CI, ¹⁴C₆H₅Br, ClCH₂¹⁴COCl and the like, are commercially available. The isotopically labeled reagents are then used in standard organic chemistry synthetic techniques to incorporate the isotope atom, or atoms, into a compound of the invention as described below.

### EXAMPLES

The invention disclosed herein is exemplified by the following preparations and examples, which should not be construed to limit the scope of the disclosure.

Abbreviations used in the description of the chemistry and in the Examples that follow are:

ACN acetonitrile
AcOH acetic acid
Boc tert-butoxycarbonyl
BBr₃ borontribromide
CuBr₂ copper (II) bromide
CH₂Cl₂, DCM dichloromethane
CH₃I, MeI iodomethane
CH₃CN acetonitrile
K₂CO₃ potassium carbonate
NaH sodium hydride
MeOH methanol

### DMF N,N-dimethylformamide

### NBS N-bromosuccinimide

X-Phos 2-Dicyclohexylphosphino-2′,4′,6′-triisopropylbiphenyl
p-TsOH para-toluene-sulfonic acid
PPA polyphosphoric acid
Pd(dppf)Cl₂ [1,1′-Bis(diphenylphosphino)ferrocene]dichloropalladium(II)
Cs₂CO₃ cesium carbonate
Et₃N triethylamine
Pd₂dba₃ Tris(dibenzylideneacetone)dipalladium(0)
Pd(OH)₂ palladium hydroxide
TFA trifluoroacetic acid
DIEA diisopropylethyl amine

### DMAP 4-Dimethylaminopyridine

### Example 1

### 1-(2-methoxyphenyl)-3-(4-(1-methyl-1H-pyrrolo[2,3-c]pyridin-3-yl)phenyl)urea

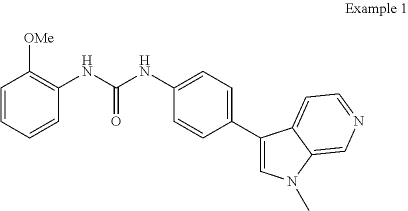 [SMILES: COc1ccccc1NC(=O)Nc1ccc(-c2cn(C)c3cnccc23)cc1]

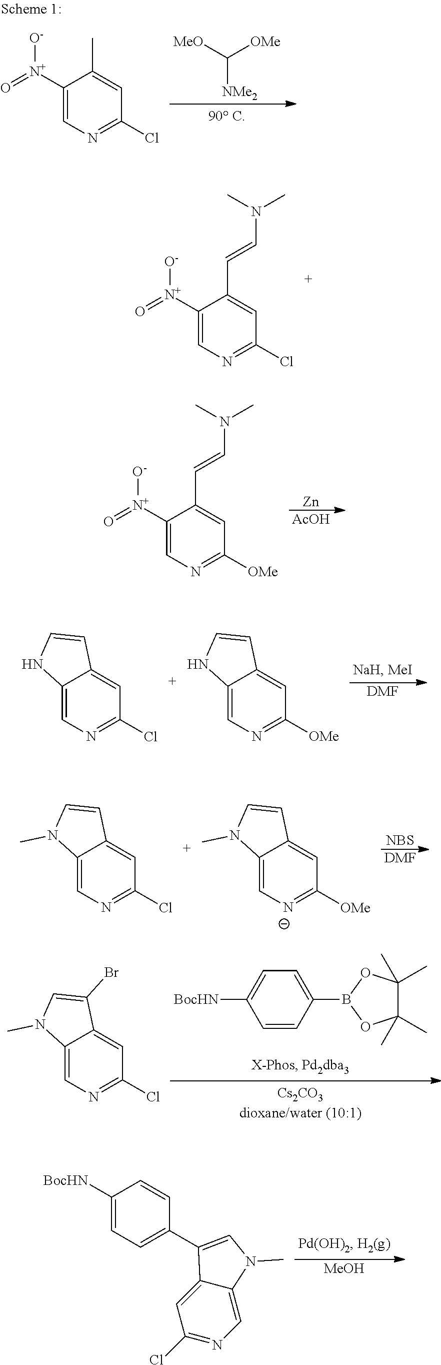

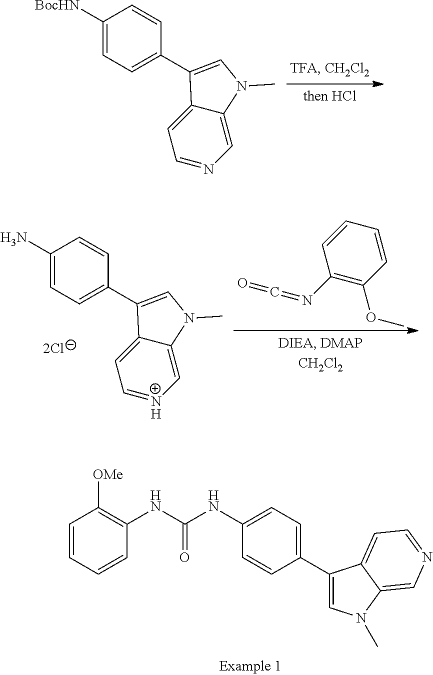 [SMILES: Cn1cc(-c2ccc(NC(=O)OC(C)(C)C)cc2)c2ccncc21 ; Cn1cc(-c2ccc(N)cc2)c2cc[nH+]cc21 ; COc1ccccc1N=C=O ; COc1ccccc1NC(=O)Nc1ccc(-c2cn(C)c3cnccc23)cc1]

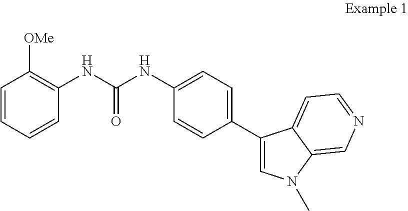 [SMILES: COc1ccccc1NC(=O)Nc1ccc(-c2cn(C)c3cnccc23)cc1]

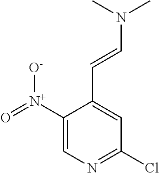

### 2-(2-chloro-5-nitropyridin-4-yl)-N,N-dimethylethenamine

2-chloro-4-methyl-5-nitropyridine (5.00 g, 29.0 mmol) in was dissolved in DMF (29.0 ml). DMF-DMA (8.53 ml, 63.7 mmol) was added and heated to 90° C. for 18 h. The reaction mixture was cooled to ambient temperature, then poured into 60 mL of water to precipitate out a solid. The mixture was filtered and the solid was washed with water. The solid was dried under high vacuum to give 5.56 g of a red powdery solid which consisted of a mixture of the title compound with approximately 9% of 2-(2-methoxy-5-nitropyridin-4-yl)-N,N-dimethylethenamine. This mixture was taken on without additional purification. LRMS (ESI) calc'd for (C₉H₁₀ClN₃O₂) [M+H]+, 228; found 228.

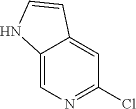

### 5-chloro-1H-pyrrolo[2,3-c]pyridine

Zinc (15.97 g, 244.0 mmol) was suspended in acetic acid (240 ml) and cooled to 0° C. Solid 2-(2-chloro-5-nitropyridin-4-yl)-N,N-dimethylethenamine (5.56 g, 24.42 mmol) was added portion-wise over about 5 minutes and then the reaction mixture was placed under an atmosphere of argon. The reaction was stirred for 18 h, after which time LCMS analysis indicated that the reaction was complete. The mixture was filtered through Celite, washing with EtOAc. The filtrate was concentrated to give a brown oil. The reaction mixture was partitioned between EtOAc and 1N NaOH. The aqueous layer was extracted with EtOAc (3×) and the combined organic layer was dried over Na₂SO₄, filtered and concentrated. The residue was adsorbed onto silica gel and purified by flash column chromatography on silica gel, eluting with EtOAc/isohexane (0-100%) to give 2.79 g of an off-white solid, that is approximately a 10:1 mixture of the title compound along with 5-methoxy-1H-pyrrolo[2,3-c]pyridine as a minor impurity. The mixture was taken forward into the next step as is. LRMS (ESI) calc'd for (C₇H₅C1N₂) [M+H]+, 153; found 153.

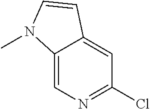

### 5-chloro-1-methyl-1H-pyrrolo[2,3-c]pyridine

A mixture of 5-chloro-1H-pyrrolo[2,3-c]pyridine (0.909 g, 5.96 mmol) and 5-methoxy-1H-pyrrolo[2,3-c]pyridine (0.089 g, 0.60 mmol) was dissolved in DMF (29.8 ml). NaH (0.357 g, 8.94 mmol) was added in one portion and the reaction mixture was stirred for 0.5 h. Methyl iodide (0.413 ml, 6.60 mmol) was added the reaction mixture was stirred for 1.0 h, after which time LCMS analysis indicated complete conversion. The reaction was quenched by the careful addition of saturated aqueous NH₄Cl. The reaction mixture was diluted in EtOAc, washed with saturated aqueous sodium hydrogen carbonate and brine then dried over Na₂SO₄ filtered and concentrated to give 1.614 g of a yellow oil. 1H-NMR and LCMS analysis indicated a mixture of the title compound along with the minor impurity (approximately 10%) 5-methoxy-1-methyl-1H-pyrrolo[2,3-c]pyridine. This mixture was taken on without additional purification. LRMS (ESI) calc'd for (C₈H₇ClN₂) [M+H]+, 167; found 167.

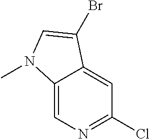

### 3-bromo-5-chloro-1-methyl-1H-pyrrolo[2,3-c]pyridine

A mixture of 5-chloro-1-methyl-1H-pyrrolo[2,3-c]pyridine (0.881 g, 5.29 mmol) and 5-methoxy-1-methyl-1H-pyrrolo[2,3-c]pyridine (0.086 g, 0.529 mmol) were dissolved in DMF (25 ml). N-bromosuccinimide (1.036 g, 5.82 mmol) was added. After 1.5 h, LCMS analysis indicated incomplete conversion so additional N-bromosuccinimide (10.0 mg, 0.058 mmol) was added. After an additional 1 h, LCMS analysis indicated the reaction was complete. The reaction mixture was diluted in EtOAc, washed with 1N aqueous sodium hydroxide and brine then dried over Na₂SO₄. The solution was filtered, concentrated, adsorbed onto silica gel, and purified by flash column chromatography on silica gel eluting with EtOAc/isohexane (10-90%) to give 1.3 g of the title compound. LRMS (ESI) calc'd for (C₈H₆⁷⁹BrClN₂) [M+H]+, 245; found 245.

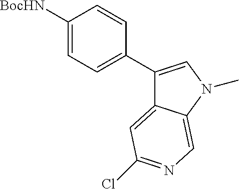 [SMILES: Cn1cc(-c2ccc(NC(=O)OC(C)(C)C)cc2)c2cc(Cl)ncc21]

### tert-butyl (4-(5-chloro-1-methyl-1H-pyrrolo[2,3-c]pyridin-3-yl)phenyl)carbamate

3-bromo-5-chloro-1-methyl-1H-pyrrolo[2,3-c]pyridine (500.0 mg, 2.04 mmol), tert-butyl (4-(4,4,5,5-tetramethyl-1,3,2-dioxaborolan-2-yl)phenyl)carbamate (683 mg, 2.14 mmol), 2-dicyclohexylphosphino-2′,4′,6′-tri-isopropyl-1,1′-biphenyl (194 mg, 0.407 mmol), tris(dibenzylideneacetone)dipalladium(0) (186 mg, 0.204 mmol), and Cs₂CO₃ (2.32 g, 7.13 mmol) were suspended in dioxane (12.3 ml)/water (1.2 ml). The mixture was sparged with argon for 10 minutes, then heated to 60° C. After 18 h, LCMS indicated complete conversion. The mixture was cooled to ambient temperature, diluted in ethyl acetate, washed with saturated aqueous sodium hydrogen carbonate and brine then dried over Na₂SO₄. The solution was filtered, concentrated, adsorbed onto silica gel, then purified by flash column chromatography on silica gel, eluting with EtOAc/isohexane (0-100%) to give the 1.694 g of the title compound as a yellow solid. LRMS (ESI) calc'd for (C₁₉H₂₀ClN₃O₂) [M+H]+, 358; found 358.

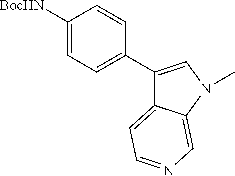 [SMILES: Cn1cc(-c2ccc(NC(=O)OC(C)(C)C)cc2)c2ccncc21]

### tert-butyl (4-(1-methyl-1H-pyrrolo[2,3-c]pyridin-3-yl)phenyl)carbamate

tert-butyl (4-(5-chloro-1-methyl-1H-pyrrolo[2,3-c]pyridin-3-yl)phenyl)carbamate (500.0 mg, 1.397 mmol) was suspended in MeOH (4 ml) under an atmosphere of argon. Palladium hydroxide on carbon (98.0 mg, 0.140 mmol) was added and the reaction mixture was placed under an atmosphere of H₂ (balloon). After 6 h, LCMS analysis indicated about 30% conversion. The flask was charged with additional MeOH (4 ml) and stirred for 15 h under an atmosphere of H₂ (balloon). At this time, LCMS analysis indicated about 50% conversion. The flask was charged with additional palladium hydroxide on carbon (98 mg, 0.140 mmol) and placed under an atmosphere of H₂ (balloon). After 6 h, LCMS analysis indicated complete conversion. The reaction mixture was filtered through a 45 μm filter and concentrated to give 480.0 mg of the title compound as a yellow solid. LRMS (ESI) calc'd for (C₁₉H₂₁N₃O₂) [M+H]+, 324; found 324.

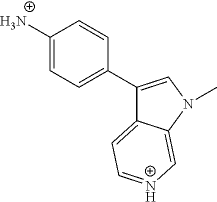 [SMILES: Cn1cc(-c2ccc([NH3+])cc2)c2cc[nH+]cc21]

### 3-(4-ammoniophenyl)-1-methyl-1H-pyrrolo[2,3-c]pyridin-6-ium

tert-butyl (4-(1-methyl-1H-pyrrolo[2,3-c]pyridin-3-yl)phenyl)carbamate (480.0 mg, 1.484 mmol) was dissolved in dichloromethane (10 ml)/TFA (1.5 ml). After 40 minutes, LCMS analysis indicated complete conversion. The reaction mixture was concentrated to give an orange oil. The oil was dissolved in MeOH and passed through a 45 μM syringe filter. HCl (4M in dioxane, a 10 mL) was added to the filtrate to give a pale yellow precipitate. The mixture was concentrated to dryness, suspended in MeOH (10 mL), and HCl (4M in dioxane, a 10 mL) was added. Concentrate and place under vacuum for 18 h to give 361 mg of the title compound as a pale yellow solid. LRMS (ESI) calc'd for (C₁₄H₁₃N₃) [M+H]+, 224; found 224.

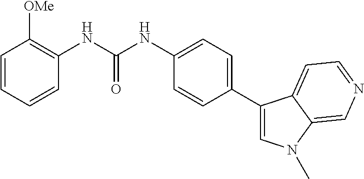 [SMILES: COc1ccccc1NC(=O)Nc1ccc(-c2cn(C)c3cnccc23)cc1]

### 1-(2-methoxyphenyl)-3-(4-(1-methyl-1H-pyrrolo[2,3-c]pyridin-3-yl)phenyl)urea

3-(4-ammoniophenyl)-1-methyl-1H-pyrrolo[2,3-c]pyridin-6-ium (100.0 mg, 0.338 mmol) was suspended in dichloromethane (4 ml). Diisopropylethylamine (0.177 ml, 1.013 mmol) was added and the reaction mixture was allowed to stir until a clear yellow homogeneous solution formed. Add 4-dimethylaminopyridine (4.1 mg, 0.03 mmol) and 2-methoxyphenyl isocyanate (50.4 mg, 0.338 mmol). After 2.5 h, LCMS analysis indicated that the reaction was nearly complete. Additional 2-methoxyphenyl isocyanate (5.0 mg, 0.034 mmol) was added. After 1 h, LCMS analysis indicated that a small amount of aniline still remained. The reaction mixture was heated to 35° C. and stirred for 18 h., at which time LCMS analysis indicated complete conversion. The mixture was concentrated to give an orange oil. The residue was purified by reverse phase (C18) preparative HPLC, eluting with Acetonitrile/Water+0.05% TFA (20-100%). The fractions containing the desired product were combined and the acetonitrile was removed under reduced pressure to give a yellow slurry in water. The slurry was diluted with 4:1 chloroform/isopropanol and saturated aqueous NaHCO₃. The heterogeneous mixture was heated to 50° C. until all of the solids dissolved. The organic layer was washed with saturated aqueous NaHCO₃and brine, then dried over Na₂SO₄ and concentrated to give 96 mg of the title compound as a yellow solid. ¹H NMR (500 MHz, DMSO-d6) δ 9.35 (s, 1H), 8.87 (s, 1H), 8.22 (s, 1H), 8.18 (d, J=5.5 Hz, 1H), 8.13 (d, J=8 Hz, 1H), 7.86 (s, 1H), 7.80 (d, J=5 Hz, 1H), 7.61-7.57 (m, 2H), 7.55-7.50 (m, 2H), 7.01 (d, J=8 Hz, 1H), 6.96-6.86 (m, 2H), 3.93 (s, 3H), 3.88 (s, 3H). LRMS (ESI) calc'd for (C₂₂H₂₀N₄O₂) [M+H]+, 373; found 376.

### Examples 2 and 3

### 1-(4-(5,6-dihydro-4H-pyrrolo[1,2-b]pyrazol-3-yl)phenyl)-3-(2-methoxyphenyl)urea (Example 2) and 1-(2-methoxyphenyl)-3-(4-(1-methyl-1H-pyrazol-4-yl)phenyl)urea

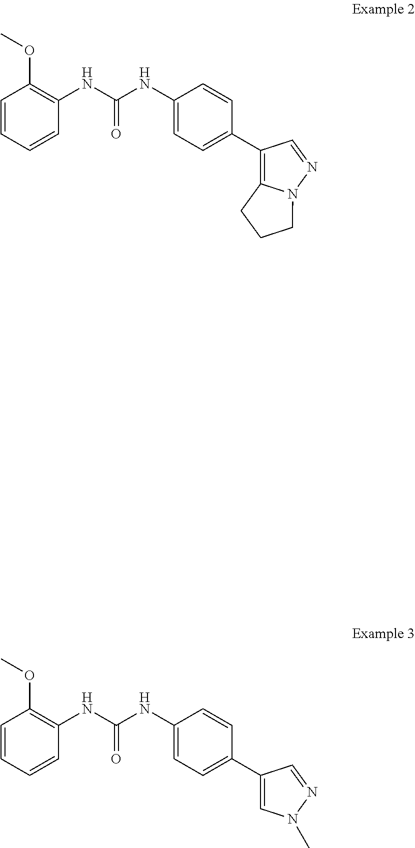 [SMILES: COc1ccccc1NC(=O)Nc1ccc(-c2cnn3c2CCC3)cc1 ; COc1ccccc1NC(=O)Nc1ccc(-c2cnn(C)c2)cc1]

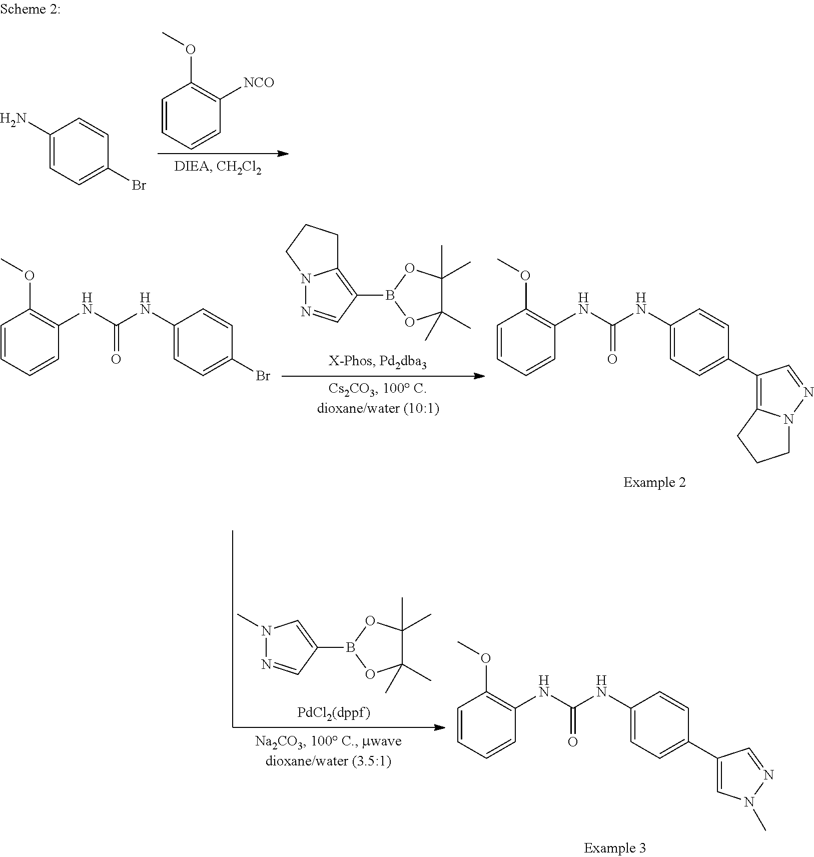

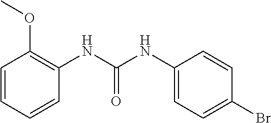 [SMILES: COc1ccccc1NC(=O)Nc1ccc(Br)cc1]

### 1-(4-bromophenyl)-3-(2-methoxyphenyl)urea

4-bromoaniline (4.00 g, 23.3 mmol) was dissolved in dichloromethane (200 mL) in a 500 ml round bottom flask. Diisopropylethylamine (4.06 mL, 23.3 mmol) was added, followed by 2-methoxyphenyl isocyanate (3.40 mL, 25.6 mmol). The flask was capped and allowed to stir overnight at ambient temperature, during which time a thick precipitate formed. The reaction mixture was filtered, the solid was washed with dichloromethane, then dried in vacuo to give the title compound. The filtrate was left to stir overnight at room temperature, during which time additional material precipitated out of solution. The mixture was filtered, washed with dichloromethane, and dried in vacuo. The first two crops provided 2.60 g of the title material. An additional 500 mg was obtained by concentrating the second filtrate and triturating with dichloromethane. LRMS (ESI) calc'd for (C₁₄H₁₃⁷⁹BrN₂O₂) [M+H]+, 321; found 321.

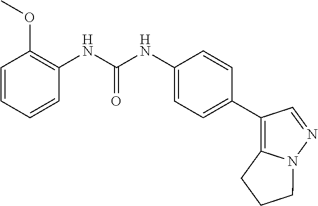 [SMILES: COc1ccccc1NC(=O)Nc1ccc(-c2cnn3c2CCC3)cc1]

### 1-(4-(5,6-dihydro-4H-pyrrolo[1,2-b]pyrazol-3-yl)phenyl)-3-(2-methoxyphenyl)urea

A flask was charged with 1-(4-bromophenyl)-3-(2-methoxyphenyl)urea (41.0 mg, 0.128 mmol), 3-(4,4,5,5-tetramethyl-1,3,2-dioxaborolan-2-yl)-5,6-dihydro-4H-pyrrolo[1,2-b]pyrazole (32.9 mg, 0.140 mmol), 2-dicyclohexylphosphino-2′,4′,6′-tri-isopropyl-1,1′-biphenyl (12.2 mg, 0.026 mmol), tris(dibenzylideneacetone)dipalladium(0) (11.7 mg, 0.013 mmol), and Cs₂CO₃ (146.0 mg, 0.447 mmol) and placed under a nitrogen atmosphere. The mixture was suspended in dioxane (1 mL)/water (1 mL) and then the reaction mixture was sparged with nitrogen for 10 minutes, and heated to 100° C. After 3 h, LCMS analysis indicated complete conversion. The reaction mixture was cooled to ambient temperature, diluted in ethyl acetate, and washed with saturated aqueous sodium hydrogen carbonate. The organic layer was separated and the aqueous layer was back extracted with EtOAc (2×). The combined organic layer was dried with Na₂SO₄, filtered and concentrated. The residue was purified by Reverse phase (C-18) preparative HPLC, eluting with Acetonitrile/Water+0.1% TFA (20-100%). The fractions containing the desired product were combined, diluted with EtOAc, washed with saturated aqueous NaHCO₃, and dried over Na₂SO₄. The solution was filtered and concentrated to give 38 mg of the title compound as a yellow solid. ¹H NMR (500 MHz, DMSO-d6) δ 9.28 (s, 1H), 8.19 (s, 1H), 8.11 (dd, J=7.8, 1.8 Hz, 1H), 7.78 (s, 1H), 7.45-7.36 (m, 4H), 7.00 (dd, J=8.3, 1.3 Hz, 1H), 6.94-6.84 (m, 2H), 4.05 (t, J=7.3 Hz, 2H), 3.86 (s, 3H), 3.03 (t, J=7.5 Hz, 2H), 2.62-2.54 (m, 2H). LRMS (ESI) calc'd for (C₂₀H₂₀N₄O₂) [M+H]+, 349; found 349.

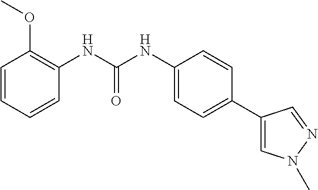 [SMILES: COc1ccccc1NC(=O)Nc1ccc(-c2cnn(C)c2)cc1]

### 1-(2-methoxyphenyl)-3-(4-(1-methyl-1H-pyrazol-4-yl)phenyl)urea

A microwave vial was charged with 1-(4-bromophenyl)-3-(2-methoxyphenyl)urea 1 (29.6 mg, 0.092 mmol), 1-methyl-4-(4,4,5,5-tetramethyl-1,3,2-dioxaborolan-2-yl)-1H-pyrazole (39.9 mg, 0.192 mmol), 2 M aqueous sodium carbonate (0.10 ml, 0.20 mmol), 1,1′-bis(diphenylphosphino)ferrocene-palladium(II)dichloride (15.1 mg, 0.021 mmol), dioxane (0.35 ml) and water (0.1 ml). The vial was sealed and heated to 100° C. for 10 minutes in a Biotage Initiator series microwave. The reaction mixture was filtered through Celite, then diluted with EtOAc and water. The organic phase was washed with brine, dried and concentrated. The residue was which was purified by flash column chromatography on silica gel, eluting with 4% MeOH/dichloromethane, followed by a second purification on silica gel, eluting with 2% MeOH/dichloromethane to afford 4.5 mg of the title compound. ¹H NMR (500 MHz, acetone-d6) δ 8.63 (s, 1H), 8.31 (dd, J=7.8, 1.8 Hz, 1H), 7.89 (s, 1H), 7.87 (s, 1H), 7.73 (s, 1H), 7.56-7.52 (m, 2H), 7.50-7.46 (m, 2H), 7.00-6.89 (m, 3H), 3.90 (s, 3H), 3.88 (s, 3H). LRMS (ESI) calc'd for (C₁₈H₁₈N₄O₂) [M+H]+, 323; found 323.

### Examples 4 and 5

### 1-(2-methoxyphenyl)-3-(6-(1-methyl-1H-pyrrolo[2,3-c]pyridin-3-yl)pyridin-3-yl)urea (Example 4) & 1-(2-hydroxyphenyl)-3-(6-(1-methyl-1H-pyrrolo[2,3-c]pyridin-3-yl)pyridin-3-yl)urea (Example 5)

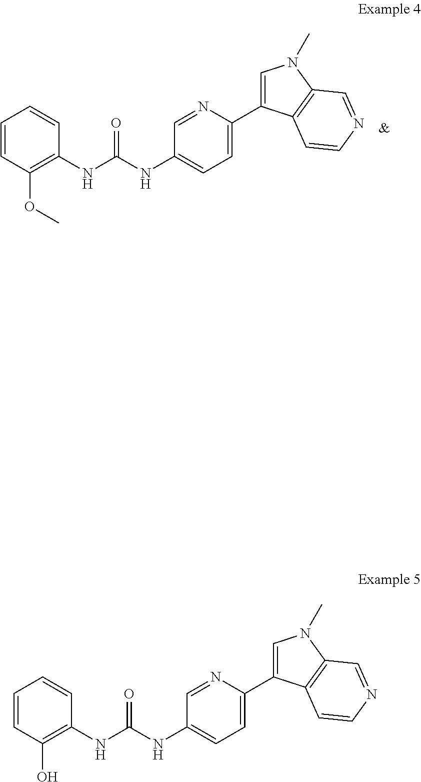 [SMILES: COc1ccccc1NC(=O)Nc1ccc(-c2cn(C)c3cnccc23)nc1 ; Cn1cc(-c2ccc(NC(=O)Nc3ccccc3O)cn2)c2ccncc21]

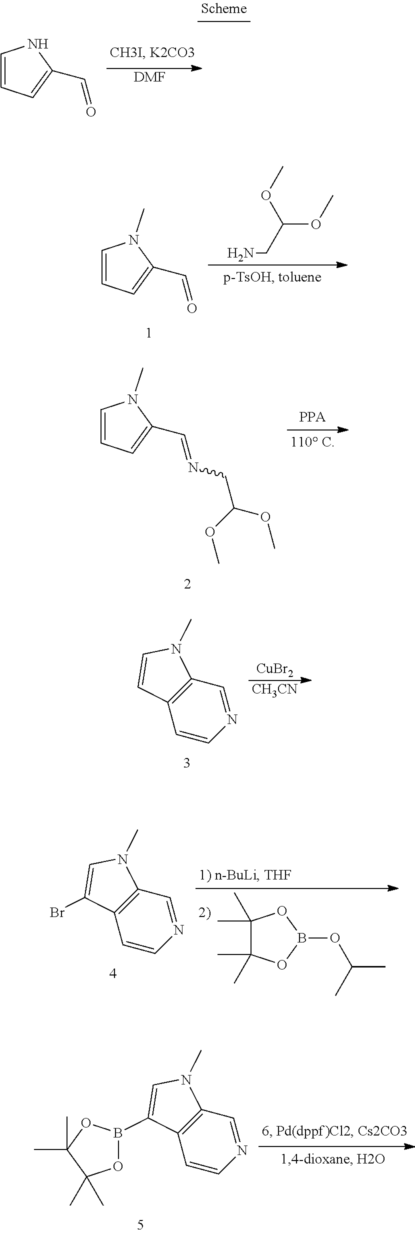

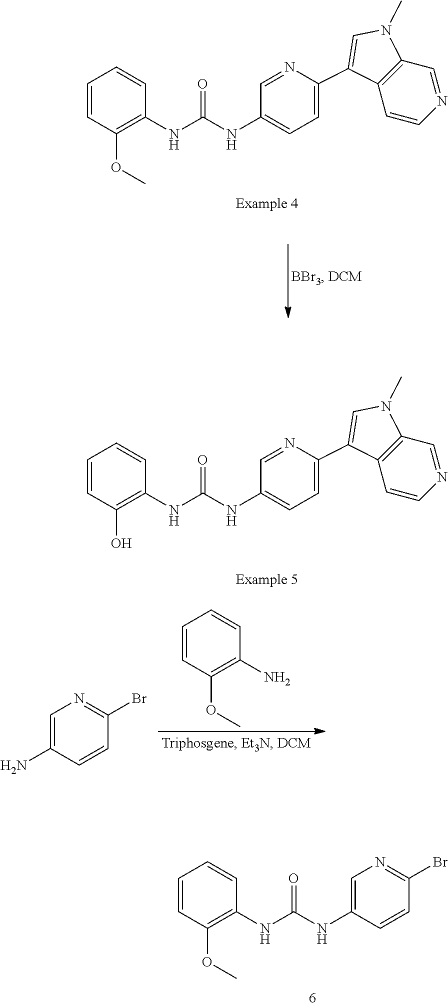

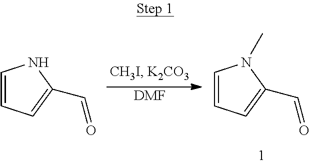

### 1-methyl-1H-pyrrole-2-carbaldehyde (1)

A mixture of 1H-pyrrole-2-carbaldehyde (50 g, 0.52 mol) and K₂CO₃ (145 g, 1.05 mol) in DMF (500 mL) was treated with CH₃I (90 g, 0.63 mol) for 10 hours at 10° C. The reaction was then quenched with water (2 L) and extracted with ethyl acetate (3×500 mL). The combined organic layer was washed with brine (4×250 mL), dried over anhydrous magnesium sulfate and concentrated under vacuum to give crude 1 as yellow oil (51 g, 89%), which is pure enough for the next step. (ES, m/z): [M+H]⁺ 110.0; ¹H NMR (300 MHz, CDCl₃) δ 9.50 (s, 1H), 6.88-6.85 (m, 2H), 6.17 (d, J=3.9 Hz, 1H), 3.89 (s, 3H).

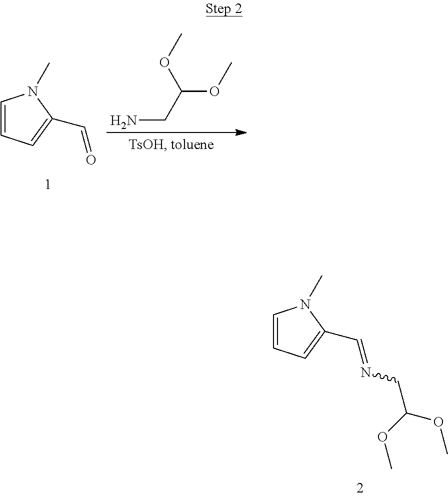

### (E,Z) 2,2-dimethoxy-N-((1-methyl-1H-pyrrol-2-yl)methylene)ethanamine (2)

A mixture of 1 (45 g, 0.42 mol), 2,2-dimethoxyethan-1-amine (60 g, 0.57 mol) and p-TsOH (0.7 g, 4 mmol) in toluene (500 mL) was heated to reflux for 4 hours while water was separated out. Then volatiles were distilled out under vacuum to give a residue, which was dissolved into dichloromethane (300 mL), washed with brine (2×50 mL), dried over anhydrous magnesium sulfate and concentrated under vacuum to give crude 2 as yellow oil (83 g), which is pure enough for the next step. (ES, m/z): [M+H]⁺ 197.0; ¹H NMR (300 MHz, CDCl₃) δ 8.09 (s, 1H), 6.67-6.66 (t, J=2.1 Hz, 1H), 6.49-6.47 (m, 1H), 6.11-6.09 (m, 1H), 4.60-4.56 (t, J=5.4 Hz, 1H), 3.89 (s, 3H), 3.64-3.62 (m, 2H), 3.38 (s, 3H), 3.36 (s, 3H).

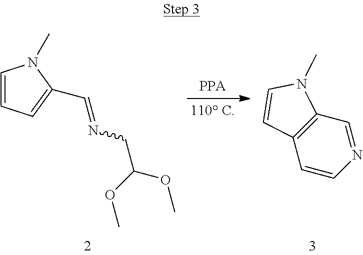

### 1-methyl-1H-pyrrolo[2,3-c]pyridine (3)

A solution of 2 (42 g, 0.21 mol) in PPA (300 g) was kept for 2 hours at 110° C. After cooled to room temperature, the reaction was quenched with water (1.5 L) and neutralized with potassium carbonate. The resulting solution was extracted with ethyl acetate (4×300 mL) and the combined organic layer was washed with brine (200 mL), dried over anhydrous magnesium sulfate and concentrated under vacuum to give a residue, which was purified by a silica gel column, eluted with 1%-2% methanol in dichloromethane to give 3 as yellow oil (14 g, 49%). (ES, m/z): [M+H]⁺ 133.0; ¹H NMR (300 MHz, CDCl₃) δ 8.76 (s, 1H), 8.24 (d, J=5.4 Hz, 1H), 7.52-7.50 ((m, 1H), 7.17 (d, J=3.0 Hz, 1H), 6.49-6.48 (m, 1H), 3.90 (s, 3H).

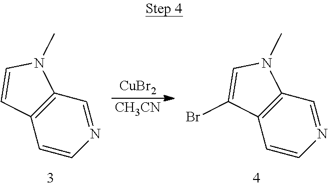

### 3-bromo-1-methyl-1H-pyrrolo[2,3-c]pyridine (4)

To a solution of 3 (14 g, 106 mmol) in CH₃CN (250 mL) was added CuBr₂ (71 g, 317 mmol). The resulting solution was stirred overnight at room temperature and then quenched by the addition of concentrated aqueous solution of ammonia (20 mL). Volatiles were distilled out under vacuum to give a residue, which was dissolved into ethyl acetate (150 mL), washed with brine (2×50 mL), dried over anhydrous sodium sulfate and concentrated under vacuum. The residue was purified by a silica gel column, eluted with 1%-2% methanol in dichloromethane to give 4 as a brown solid (11 g, 49%). (ES, m/z): [M+H]⁺ 211.0 and 213.0; ¹H NMR (300 MHz, CDCl₃) δ 8.77 (br s, 1H), 8.36 (br s, 1H), 7.46 (m, 1H), 7.21 (s, 1H), 3.89 (s, 3H).

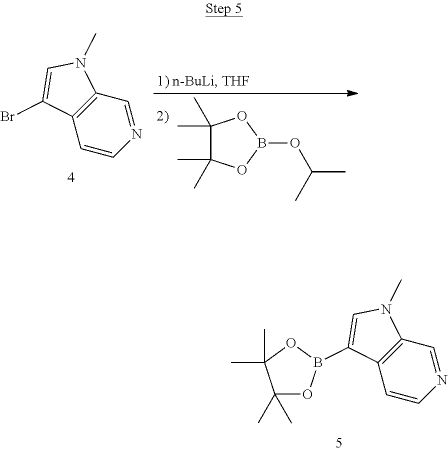

### 1-methyl-3-(4,4,5,5-tetramethyl-1,3,2-dioxaborolan-2-yl)-1H-pyrrolo[2,3-c]pyridine (5)

A solution of 4 (11 g, 52 mmol) in dry THF (150 mL) was treated with n-BuLi (33 mL, 83 mmol, 2.5 M) for 30 min at −78° C. followed by the addition of 4,4,5,5-tetramethyl-2-(propan-2-yloxy)-1,3,2-dioxaborolane (12.6 g, 68 mmol) in THF (25 mL). After additional 3 hours at −50° C., the reaction was then quenched by the addition of saturated aqueous NH₄Cl solution (500 mL). The resulting solution was extracted with ethyl acetate (3×100 mL) and the combined organic layer was washed with brine (2×50 mL), dried over anhydrous sodium sulfate and concentrated under vacuum. The residue was purified by a silica gel column, eluted with 20%-60% ethyl acetate in petroleum ether to give 5 as a white solid (8 g, 59%). (ES, m/z): [M+H]⁺ 259.0; ¹H NMR (300 MHz, CDCl₃) δ 8.75 (br s, 1H), 8.34 (br s, 1H), 7.45 (m, 1H), 7.20 (s, 1H), 3.89 (s, 3H), 1.32 (br s, 12H).

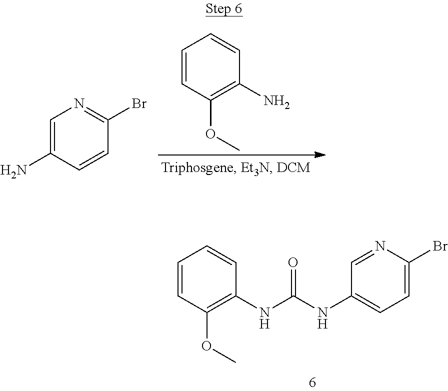

### 1-(6-bromopyridin-3-yl)-3-(2-methoxyphenyl)urea (6)

To a solution of triphosgene (5.7 g, 19 mmol) in dry dichloromethane (70 mL) was added a mixture of 6-bromopyridin-3-amine (10 g, 57.8 mmol) and triethylamine (7 g, 69 mmol) in dichloromethane (30 mL) dropwise with stirring at 0° C. The resulting mixture was stirred for 30 min at 0° C. followed by the addition of a solution of 2-methoxyaniline (7.9 g, 64 mmol) in dichloromethane (30 mL) dropwise. After additional 2 hours at room temperature, the reaction was then quenched with water (50 mL). Solids were collected by filtration, washed with dichloromethane (3×100 mL) and water (3×100 mL), dried in an vacuum oven to give 6 as a purple solid (12 g, 64%). (ES, m/z): [M+H]⁺ 322.0; ¹H NMR (300 MHz, DMSO) δ 9.63 (s, 1H), 8.47 (s, 1H), 8.35 (s, 1H), 8.09 (d, J=7.8 Hz, 1H), 7.83 (d, J=7.8 Hz, 1H), 7.58-7.53 (m, 1H), 7.04-6.88 (m, 3H), 3.88 (s, 3H).

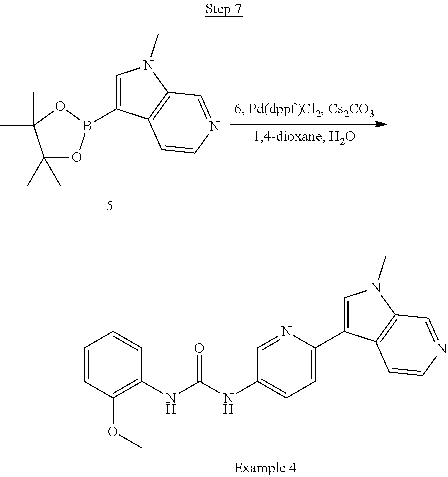 [SMILES: Cn1cc(B2OC(C)(C)C(C)(C)O2)c2ccncc21 ; COc1ccccc1NC(=O)Nc1ccc(-c2cn(C)c3cnccc23)nc1]

### 1-(2-methoxyphenyl)-3-(6-(1-methyl-1H-pyrrolo[2,3-c]pyridin-3-yl)pyridin-3-yl)urea

### Example 4

A mixture of 5 (201 mg, 0.55 mmol), 6 (160 mg, 0.5 mmol), Pd(dppf)Cl₂ (73 mg, 0.1 mmol) and Cs₂CO₃ (488 mg, 1.5 mmol) in 1,4-dioxane (15 mL) and H₂O (1 mL) was kept for 5 hours at 90° C. under nitrogen atmosphere, then the reaction was quenched with water (50 mL). The solids were collected by filtration and purified by a silica gel column, eluted with 1%-5% methanol in dichloromethane to give Example 4 as a yellow solid (127.3 mg, 69%). (ES, m/z): [M+H]⁺ 374.0; ¹H NMR (300 MHz, DMSO) δ 9.53 (br s, 1H), 8.87 (s, 1H), 8.60 (s, 1H), 8.34 (br s, 1H), 8.26-8.11 (m, 4H), 8.01-7.98 (m, 1H), 7.76-7.73 (m, 1H), 7.01-6.87 (m, 3H), 3.96 (s, 3H), 3.88 (s, 3H).

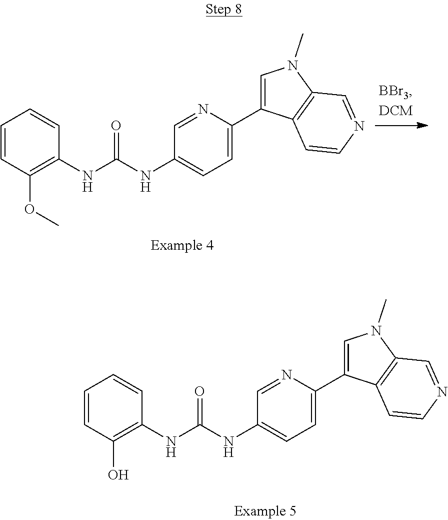 [SMILES: COc1ccccc1NC(=O)Nc1ccc(-c2cn(C)c3cnccc23)nc1 ; Cn1cc(-c2ccc(NC(=O)Nc3ccccc3O)cn2)c2ccncc21]

### 1-(2-hydroxyphenyl)-3-(6-(1-methyl-1H-pyrrolo[2,3-c]pyridin-3-yl)pyridin-3-yl)urea

### Example 5

A suspension of Example 4 (300 mg, 0.8 mmol) in dichloromethane (20 mL). was treated with tribromoborane (1 g, 4 mmol) at room temperature for 10 hours. The reaction quenched with methanol (10 mL) and neutralized with concentrated aqueous ammonia. The solids were collected by filtration and washed with water (3×10 mL) to give Example 5 as a light yellow solid (210 mg, 73%). (ES, m/z): [M+H]⁺ 360.0; ¹H NMR (300 MHz, CD₃OD) δ 9.44 (s, 1H), 9.23 (d, J=1.8 Hz, 1H), 8.77 (s, 1H), 8.65 (d, J=4.8 Hz, 1H), 8.47 (d, J=4.8 Hz, 1H), 8.32 (dd, J₁=0.9 Hz, J₂=6.6 Hz, 1H), 8.24 (d, J=14.7 Hz, 1H), 8.00 (dd, J₁=0.9 Hz, J₂=3.0 Hz, 1H), 6.95-6.75 (m, 3H), 4.26 (s, 3H)

### Example 6

### Preparation of [³H]-(2-methoxyphenyl)-3-(4-(1-methyl-1H-pyrrolo[2,3-c]pyridin-3-yl)phenyl)urea

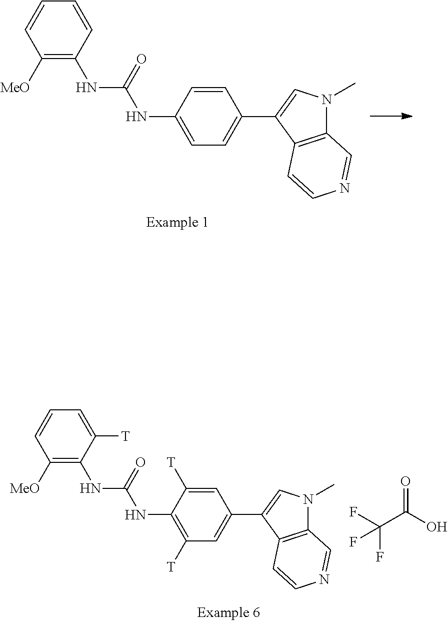

To a trisorber reaction vessel (total volume ˜2 ml) with stir bar was added Crabtree's catalyst (8.6 mgs, 2 eq) and a solution of Example 1 (2.1 mg, 5.64 μmol) in ˜1.3 ml DCM. The vessel was connected to the Trisorber manifold (product of INUS/Lablogic) cooled in dry ice/EtOH, degassed, filled with T₂ (450-14 torr) and stirred at RT 0/N. The reaction was re-cooled, degassed and concentrated in vacuo. LCMS of the crude material shows product with SA of 61.6 Ci/mmol. The crude material was purified by RP HPLC (Luna C18(2), 5u, 10×250 mm, 5 ml/min, 32% ACN/H2O+0.1% TFA, pda detector, Rₜ on analytical column @ 35% ACN was 9.6 min) to give (after C18 sep pak solvent switch—sep pak conditioned with EtOH and then water) 63.8625 mCi in 19.3 ml EtOH (=3.3089 mCi/m1) @ 61.3119 Ci/mmol (162.7835 mCi/mg). Deliver 19.2 ml=63.532 mCi with radiochemical purity >98.8% as a TFA salt.

### Example 7

### Preparation of [³H] 1-(2-methoxyphenyl)-3-(6-(1-methyl-1H-pyrrolo[2,3-c]pyridin-3-yl)pyridin-3-yl)urea

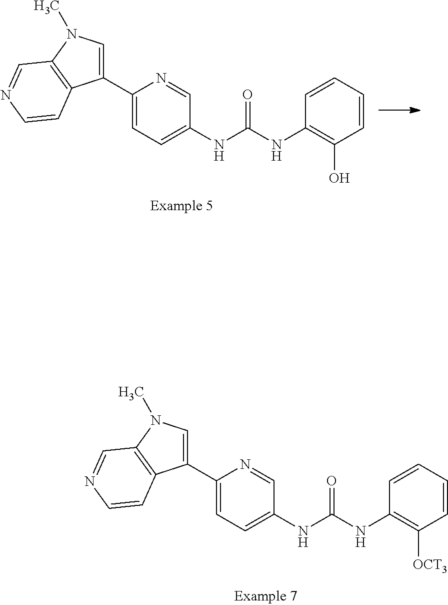

To a 2 ml HPLC vial with stir bar was added Example 5 (2.5 mg, 6.96 μmol), Cs₂CO₃ (5-7 mgs) and 0.15 ml DMF. To this was added 100 mCi ampule of CT3I (washed ampule with 0.1 ml DMF and add to reaction) to form a mixture which was stirred at room temperature for 1.5 hrs. LCMS of the crude material shows product formation and an SA of 73.55 Ci/mmol. The reaction was diluted with ACN and EtOH, filtered and concentrated in vacuo. The crude was purified by RP HPLC (Curosil PFP, 5u, 10×250 mm, 5 ml/min, 45% ACN/55% H2O+20 mM NH4OAc, pda detector, Rₜ on analytical=9 min @ 50% ACN, Rt on semi-prep=15 min @ 45% ACN) to give (after C18 sep pak solvent switch—sep pak conditioned with EtOH and then water) 11.6253 mCi in 9.4 ml EtOH (=1.2367 mCi/ml) @ 75.2501 Ci/mmol (198.7525 mCi/mg). Delivered 9.3 ml=11.502 mCi with radiochemical purity >97.5%.

### Example 8

### Preparation of In Vitro Assembled Tau Filaments

In Vitro assembled tau filaments were prepared similarly to Barghorn et al, Methods Mol Biol. 2005, 299, 35-51.

Briefly, Twenty micromolar (uM) of full length tau monomer (4R2N, T40 isoform), was incubated at 37 degrees shaking for 14 days with 50 uM heparin, 2 mM DTT, and 0.04% sodium azide in 100 mM Sodium Acetate (NaOAC) buffer pH7.0. All concentrations listed are final concentrations. Samples of the mixture were taken at 0, 10, and 14 days, and filament formation was examined using Thioflavin T binding compared to a control sample of filaments.

### Example 9

### In Vitro Binding of Tau Tracers to In Vitro Assembled Tau filaments

### Saturation Binding Assay

For the Saturation Binding assay, various concentrations of radioligand were used, ranging from 1.5 nM to 30 nM for [³H]1-(2-methoxyphenyl)-3-(6-(1-methyl-1H-pyrrolo[2,3-c]pyridin-3-yl)pyridin-3 3-yl)urea (Example 7 compound) and for 0.87 nM to 50 nM for [³H] 1-(2-methoxyphenyl)-3-(4-(1-methyl-1H-pyrrolo[2,3-c]pyridin-3-yl)phenyl)urea (Example 6 compound). Total binding was defined in the absence of competing compound, and non-displaceable binding was determined in the presence of 1 μM unlabeled self block.

Either DMSO or Cold Compound (Example 6 compound or Example 7 compound) (100× concentrated) was added to the assay plate, then either 0.4 uM In Vitro Assembled tau filaments diluted in assay buffer (PBS+0.1% BSA) or assay buffer alone was added to the assay plate.

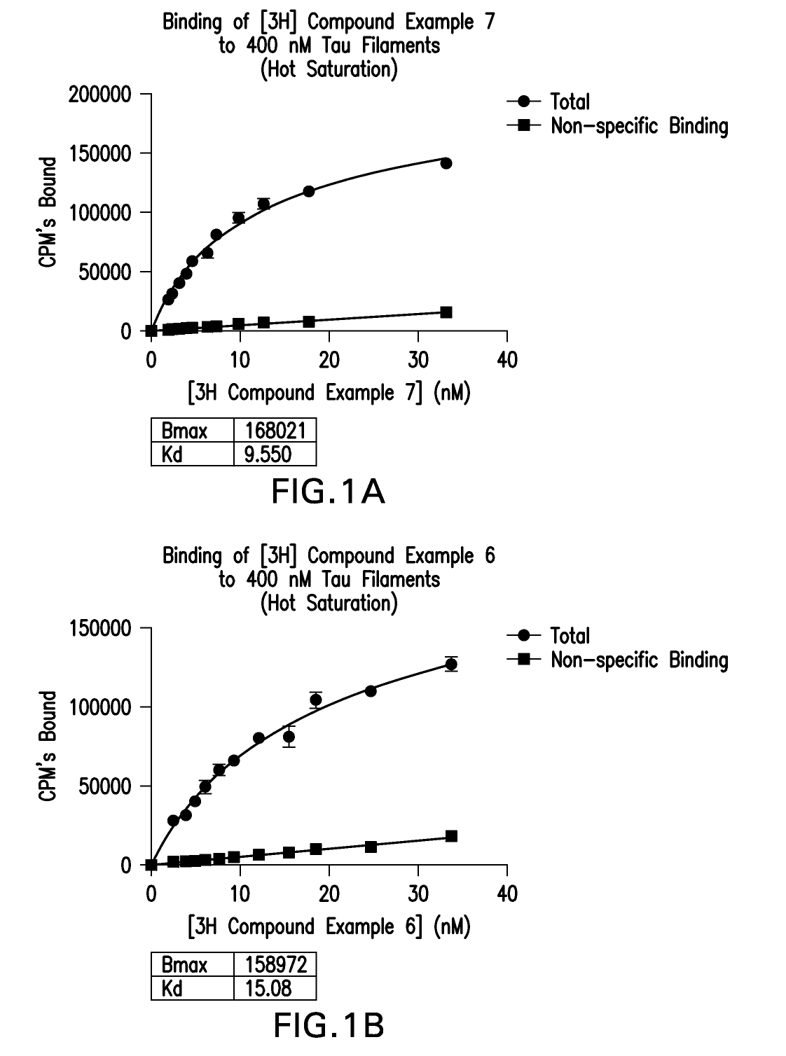

Dilutions of hot ligand were made by serial dilution in a PCR plate then added to the assay plate. Plate was covered and incubated at room temperature (25° C.) for 90 minutes with shaking. The samples on the assay plate were filtered onto GF/B filter plates (blocked at least 1 hour with 50 uL 0.2% PEI) using a PerkinElmer Filtermate 96-well plate harvester, washing 6 times with ice cold buffer (5 mM Tris, pH 7.4). Plates were dried in a vacuum oven at 37° C. for 1 hour, then plates were sealed on the back, and 50 ul of Microscint-20 was added to each well. The tops of the plates were sealed, and then plates were read in a PerkinElmer TopCount. Data analysis was performed using Graphpad Prism software using the Saturation Binding: One site analysis methods. FIGS. 1A and 1B show the calculated binding site densities (Bmax) and binding affinity (Kd) of [³H] compound of Example 7 (FIG. 1A) and [³H] compound of Example 6 (FIG. 1B) to in vitro assembled tau filaments from non-linear regression methods.

### Displacement Binding Assay

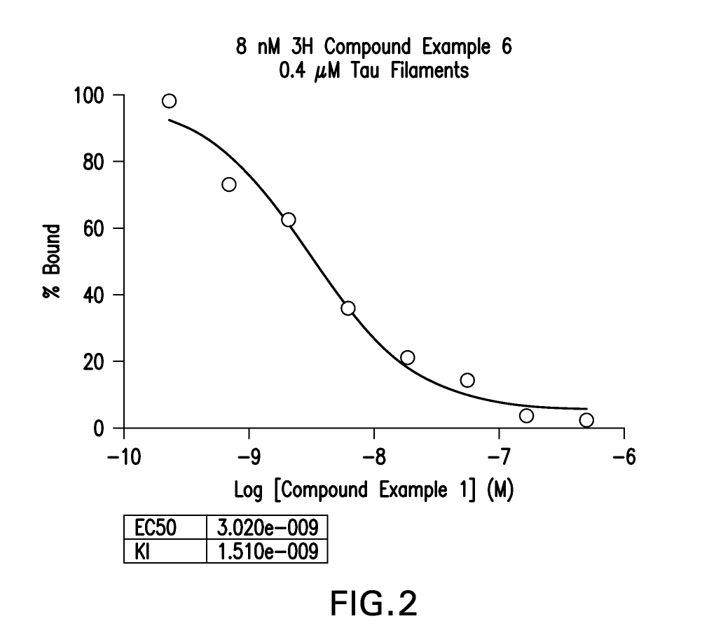

For the displacement binding assay, compound dilutions (1000×) were added into the 96-well plate (150 nL per well). 100% (1 uM self-block (unlabeled Compound—Example 1 or Example 4 dependent on the ³H-labeled input compound—Example 6 or 7, respectively)) and 0% (DMSO) controls were included in each assay plate. In Vitro Assembled Tau filaments diluted in assay buffer (PBS+0.1% BSA) were added at a final concentration of 0.4 uM per well. Subsequently, either ³H compound of Example 7 or ³H compound of Example 6 was added to each well at a final concentration of 8 nM. Incubation was carried out at room temperature (25° C.) for 90 minutes, and then the assay samples were filtered onto GF/B filter plates that had been blocked at least 1 hour with 50 uL 0.2% PEI using a PerkinElmer Filtermate 96-well plate harvester, washing 6 times with ice cold buffer (5 mM Tris, pH 7.4). Plates were dried in a vacuum oven at 37° C. for 1 hour, then plates were sealed on the back, and 50 ul of Microscint-20 was added to each well. The tops of the plates were sealed, and then plates were read in a PerkinElmer TopCount. Data analysis was performed using internally developed software (Assay Data Analyzer). FIG. 2 shows dose-dependent inhibition of [³H] compound of Example 6 binding to in vitro assembled filaments by self-block (unlabeled compound—Example 1), with an apparent IC50 of 3.0 nM. The same analysis was performed with [³H]compound of Example 7 with self unlabeled compound—Example 4, with an apparent IC50 of 20.2 nM The displacement assay using [³H]compound of Example 7 was used to identify IC50 values of experimental compounds as show in the Table below.

TABLE

| Example- | IC₅₀ |
|---|---|
| Compound | (nM) |
| 1 | 13.6 |
| 2 | 25.1 |
| 3 | 33.6 |
| 4 | 20.2 |
| 5 | 92.0 |

### Example 10

### In Vitro Binding of Tau Tracers in Human AD Brain Tissue (Sections and Homogenates)

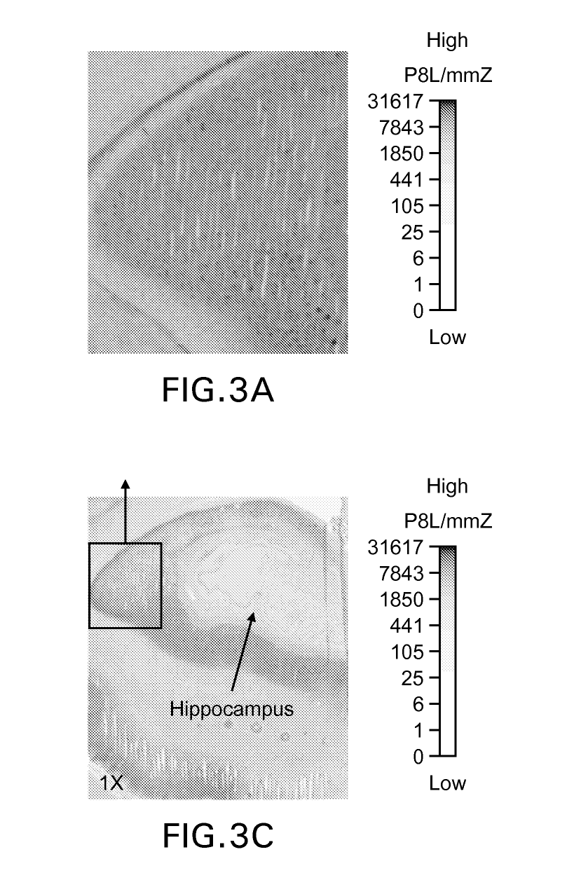

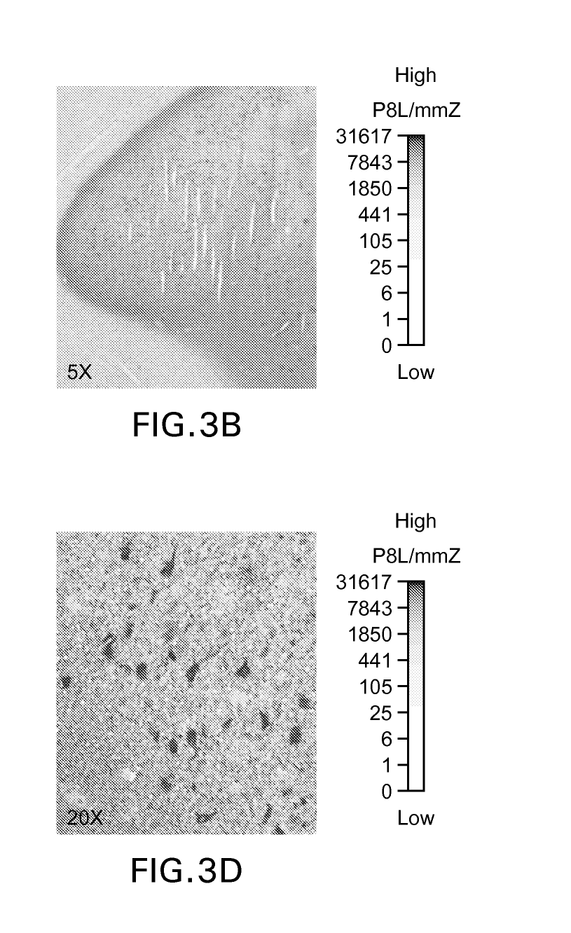

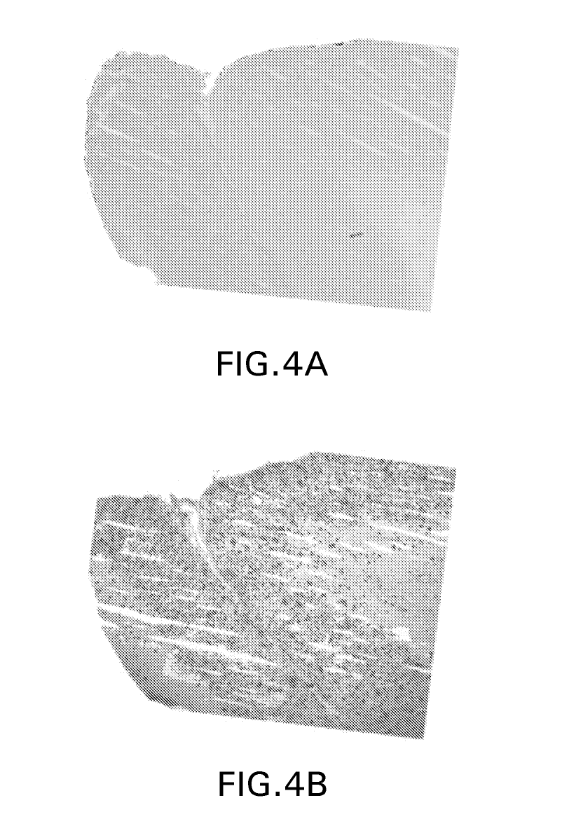

To assess presence of amyloid plaques and NFTs in the tested human brain samples, the adjacent human AD brain slices were used for autoradiography (ARG) and immunohistochemistry (IHC) studies. ARG was done using isotopically labeled compounds which bind selectively to amyloid plaques or NFTs. IHC was performed with antibodies for amyloid-beta (Aβ) (6E10 (Covance)) and phosphorylated tau (p-tau) (PHF6 (Covance)). Tissue homogenate binding was performed using human AD brain homogenates of cerebral cortex. Human brains from donor without neurological disorder were used as control in the same study. FIG. 3 shows ARG and IHC images. FIG. 3A, ³H compound of Example 6 binding in hippocampus region of human Alzheimer's disease brain slice. FIGS. 3B-3D, Images from different magnification showing positive PHF6 stain of NFTs using the adjacent brain slice of ARG study. FIG. 4 shows ARG and IHC images of human AD brain cortex. FIG. 4A, lack of [³H] compound of Example 6 binding to amyloid plaques in cortex region. FIG. 4B, the adjacent human Alzheimer's disease brain cortex slice shows positive stain of dense amyloid plaques (Abeta) by immunhistochemistry using 6E10 antibody. In sum, the ARG evidence shown in FIGS. 3 and 4 indicates that there is measurable specificity of Compound of Example 6 for Tau over Aβ.

### Procedures of In Vitro Autoradiography:

The frozen human brain samples of Alzheimer's disease (AD) and non-AD were purchased from Analytic Biological Services Inc. Frozen brain slices (20 μm thickness) were prepared using a cryostat (Leica CM3050) and kept in sequential order. The tissue slices were placed on Superfrost Plus glass slides (Cat.#5075-FR, Brain Research Laboratories, USA), dried at room temperature, and stored in a slide box at −70° C. before use. [³H]compound of Example 6 and [³H]compound of Example 7 were synthesized by Radio Compound Labelling Synthesis Group at Merck. The specific activities of [³H]compound of Example 6 and [³H]compound of Example 7 were 61.9 Ci/mmol (2.01 mCi/mL) and 75.1 Ci/mmol (2.05 mCi/mL), respectively. The final concentrations of radioligand for in vitro autoradiography were 2 nM or 6 nM with [³H]compound of Example 6, and 10.0 nM with [³H]compound of Example 7. On the day of a binding experiment, adjacent slices were selected from each brain region of interest for in vitro autoradiographic study, and were designated as total binding and non-specific binding (NSB). These slices were thawed at room temperature for 15 minutes in a biosafety hood. A single concentration of [³H]compound of Example 6 or [³H]compound of Example 7 was applied in the study. Total binding of radioligand in a brain slices was defined in the absence of competitor, and non-specific binding (NSB) was determined in the presence of competitor (1.0 μM unlabeled self block). The brain slides were first pre-incubated at room temperature for twenty minutes in PBS buffer, pH 7.4. The slices were then transferred to fresh buffer containing radioligand or radioligand plus competitor as described above, and incubated at room temperature for ninety minutes. Incubation was terminated by washing the slices three times in ice cold (4° C.) wash buffer (PBS, pH 7.4) with each wash lasting three minutes. After washing, the slices were briefly rinsed in ice cold (4° C.) deionized water, and then dried completely by an air blower at room temperature. The slices were placed against Fuji Phosphor Image Plates (TR25, Fuji) in a sealed cassette for exposure at room temperature. After one week exposure, the plates were scanned in Fuji BAS 5000 Scanner, and the scanned images were analyzed using MCID 7.0 software. [³H]-microscales (Amersham Biosciences, GE), were used for quantification of radioligand binding density. FIGS. 3 and 4 have ARG images detected through this method.

### Procedures of Tissue Homogenate Binding:

The frozen human brain samples of Alzheimer's disease (AD) and non-AD were purchased from Analytic Biological Services Inc. They were postmortem tissue from donors with clinical diagnosis of AD or non-AD. Brain homogenates of frontal cortex were prepared by homogenizing the frontal cortex in ice cold Phosphate Buffered Saline (PBS), pH 7.4, for 30 seconds at 4° C. on setting 6 of Polytron. The final concentration of brain homogenates was 10 mg wet tissue per 1 mL buffer. Homogenates were aliquoted in 5 mL/tube and stored at −70° C. prior to use.

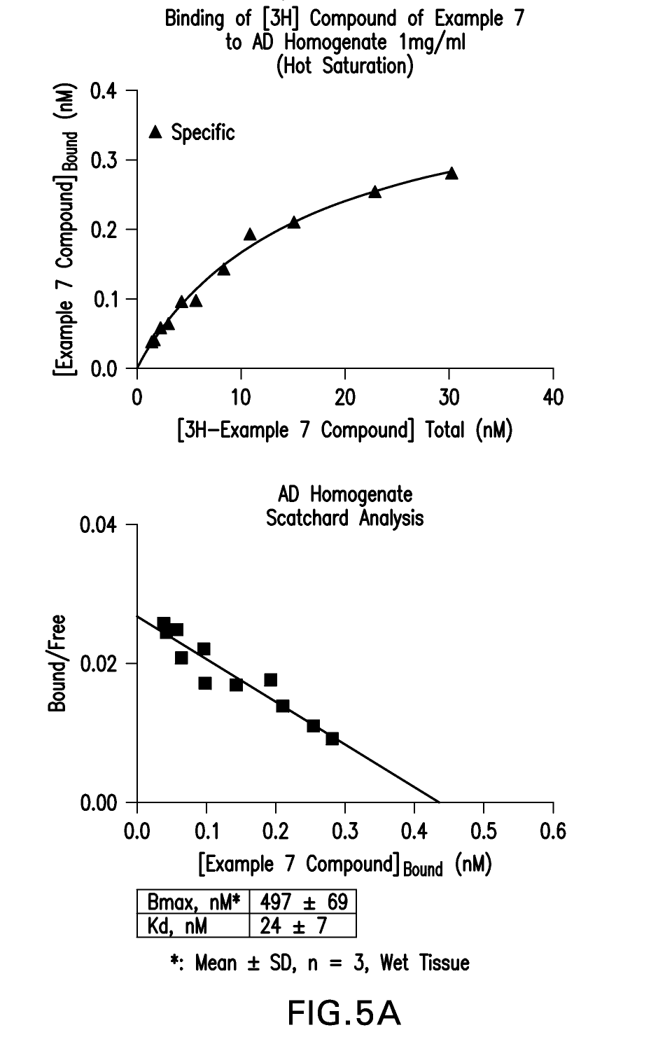

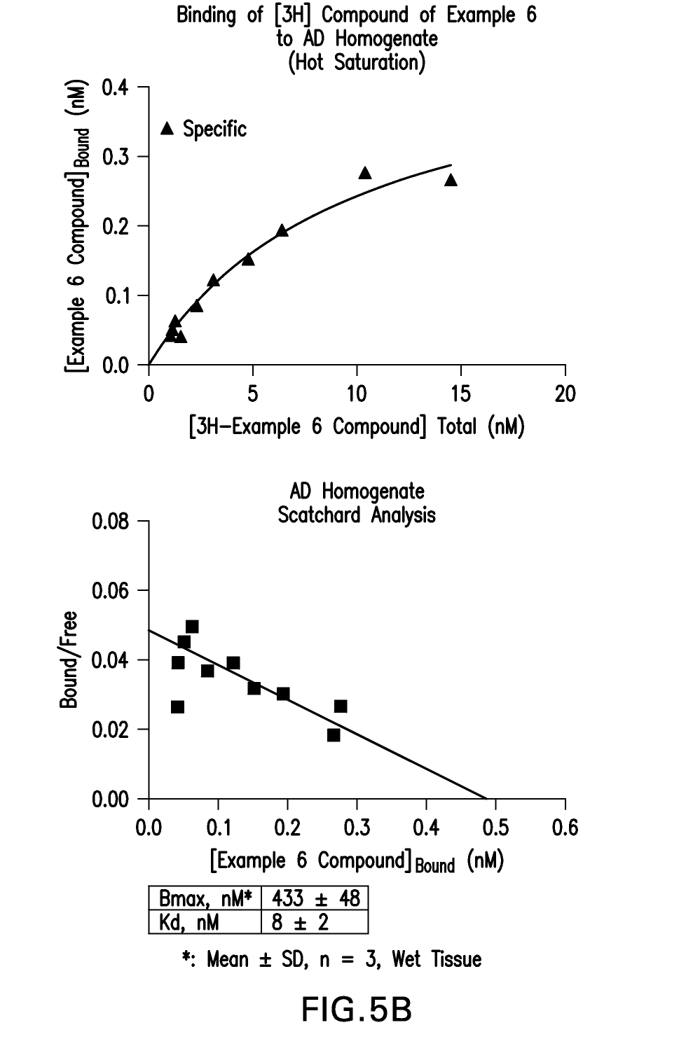

For hot saturation binding assay, various concentrations of radioligand were used, ranging from 1.5 nM to 30 nM for [³H]compound of Example 7, and from 1.1 nM to 14.5 nM for [³H]compound of Example 6. For displacement binding assay, final [³H]compound of Example 7 concentration was 2 nM. Brain homogenates were diluted to 1.0 mg/mL from original 10 mg/mL volume, with PBS buffer, and 200 μl was used in assay for a final concentration of 200 μg/assay tube. Unlabeled test compounds were dissolved in DMSO at 1 mM. Dilution of test compound to various concentrations was made with PBS containing 2% DMSO. Total binding was defined in the absence of competing compound, and non-displaceable binding was determined in the presence of 1 μM unlabeled self block. Compound dilutions (10×) were added into the assay tube (25 μL each/per tube, separately) containing 200 μL brain homogenate dilution, and the tubes were pre-incubated at room temperature for 10 minutes, then radioligand dilutions (10×) were added into the assay tube (25 μL each/per tube, separately) to a final volume of 250 μL per tube. Incubation was carried out at room temperature (25° C.) for 90 minutes, and then the assay samples were filtered onto GF/C filters using Skatron 12 well harvester, washing on setting 5−5−5 (˜3×2 ml) ice cold buffer (PBS, pH 7.4). GF/C filter papers for Skatron harvester were pre-soaked in 0.1% BSA for 1 hour at room temperature before use. Filters were punched into scintillation vials and counted in 2 mL Ultima Gold on Perkin Elmer Tri-Carb 2900TR for 1 minute. The data analysis was done with Prism software. FIG. 5A show hot saturation binding of [³H]compound of Example 6 and [³H]compound of Example 7 in brain homogenates of human Alzheimer's disease (AD) donor. Both compounds show high affinity for tau in AD brain homogenates, with measured dissociation constants (Kd) of 24 and 8 nm for [³H]compound of Example 7 and [³H]compound of Example 6, respectively. FIG. 5B shows dose-dependent inhibition of [3H] binding in AD homogenates by self block (the corresponding unlabeled compound).

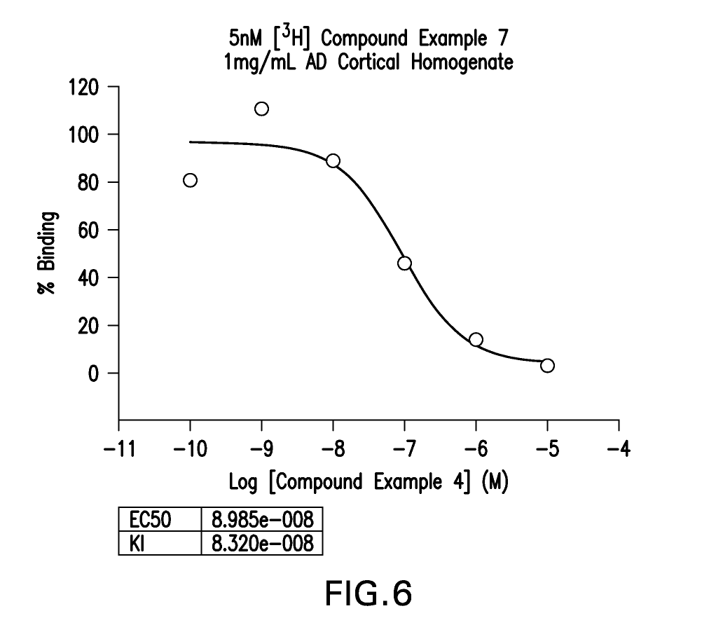

As shown in FIG. 6, Example 4 compound (unlabeled) self-displaced [³H]compound of Example 7 with an IC50 value of 89.85 nM and Ki of 83.20 nM.

What is claimed:

1. A compound of the Formula (I) 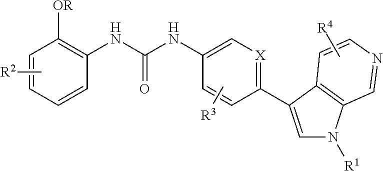
or a pharmaceutically acceptable salt thereof,
wherein
X is N or C;
R is H or C1-6alkyl optionally substituted with one fluoro;
R¹ is H or C1-6alkyl optionally substituted with one fluoro; and
R², R³ and R⁴ are each independently H, fluoro or C1-6alkyl optionally substituted with one fluoro.

2. The compound according to claim 1 or a pharmaceutically acceptable salt thereof, wherein the compound is isotopically labeled with an isotope selected from the group consisting of ²H, ³H, ¹¹C, ¹³C, ¹⁴C, and ¹⁸F.

3. The compound according to claim 1 or a pharmaceutically acceptable salt thereof, wherein the compound is selected from the group consisting of 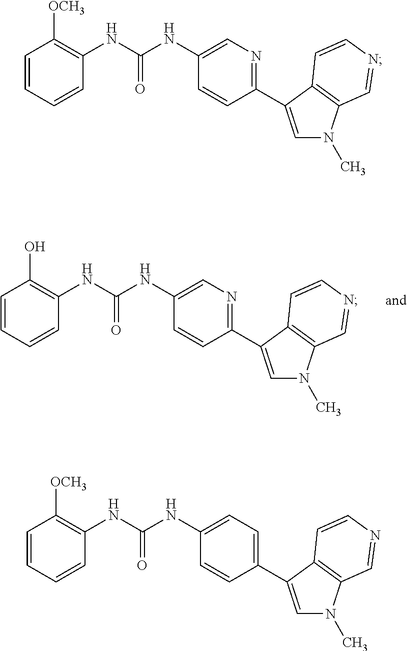 [SMILES: COc1ccccc1NC(=O)Nc1ccc(-c2cn(C)c3cnccc23)nc1 ; Cn1cc(-c2ccc(NC(=O)Nc3ccccc3O)cn2)c2ccncc21 ; "and" ; COc1ccccc1NC(=O)Nc1ccc(-c2cn(C)c3cnccc23)cc1]
or a pharmaceutically acceptable salt thereof.

4. The compound according to claim 3 or a pharmaceutically acceptable salt thereof, wherein the compound is isotopically labeled with an isotope selected from the group consisting of ²H, ³H, ¹¹C, ¹³C, and ¹⁴C.

5. The compound according to claim 4 or a pharmaceutically acceptable salt thereof, wherein the isotope is ³H.

6. The compound according to claim 5, wherein the compound is 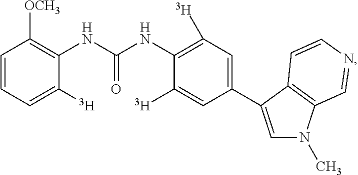 [SMILES: [3H]c1cc(-c2cn(C)c3cnccc23)cc([3H])c1NC(=O)Nc1c([3H])cccc1OC]
or a pharmaceutically acceptable salt thereof.

7. The compound according to claim 5, wherein the compound is 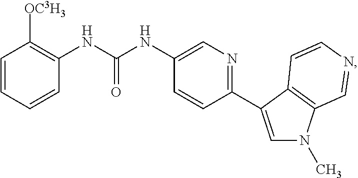
or a pharmaceutically acceptable salt thereof.

8. A pharmaceutical composition comprising the compound of claim 1 or a pharmaceutically acceptable salt thereof and a pharmaceutically acceptable carrier.

9. A pharmaceutical composition comprising the compound of claim 2 or a pharmaceutically acceptable salt thereof and a pharmaceutically acceptable carrier.

10. A compound of the Formula (II) 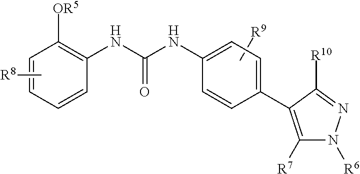
or a pharmaceutically acceptable salt thereof, wherein
R⁵ is H or C1-6alkyl optionally substituted with one fluoro;
R⁶ is H or C1-6alkyl optionally substituted with one fluoro;
R⁷ is H, fluoro or C1-6alkyl optionally substituted with one fluoro; or
R⁶ and R⁷ together complete a 5-6-membered saturated heterocyclic ring containing 4-5 carbon atoms; and
R⁸, R⁹ and R¹⁰ are each independently H, fluoro or C1-6alkyl optionally substituted with one fluoro.

11. The compound according to claim 10 or a pharmaceutically acceptable salt thereof, wherein the compound is isotopically labeled with an isotope selected from the group consisting of ²H, ³H, ¹¹C, ¹³C, ¹⁴C, and ¹⁸F.

12. The compound according to claim 10 or a pharmaceutically acceptable salt thereof, wherein the compound is selected from the group consisting of 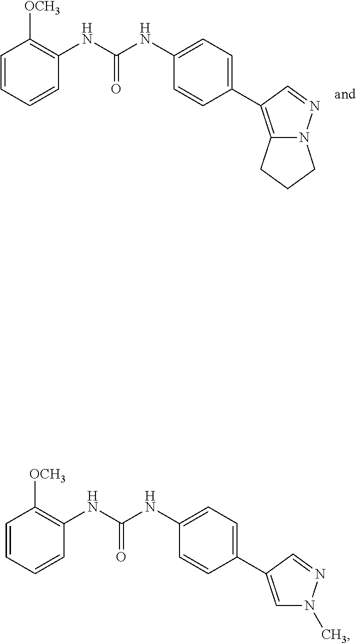 [SMILES: COc1ccccc1NC(=O)Nc1ccc(-c2cnn3c2CCC3)cc1 ; COc1ccccc1NC(=O)Nc1ccc(-c2cnn(C)c2)cc1]
or a pharmaceutically acceptable salt thereof.

13. The compound according to claim 12 or a pharmaceutically acceptable salt thereof, wherein the compound is isotopically labeled with an isotope selected from the group consisting of ²H, ³H, ¹¹C, ¹³C and ¹⁴C.

14. The compound according to claim 13 or a pharmaceutically acceptable salt thereof, wherein the isotope is ³H.

15. A pharmaceutical composition comprising the compound of claim 10 or a pharmaceutically acceptable salt thereof and a pharmaceutically acceptable carrier.

16. A pharmaceutical composition comprising the compound of claim 11 or a pharmaceutically acceptable salt thereof and a pharmaceutically acceptable carrier.
